(19) United States
(12) Patent Application Publication
(10) Pub. No.: US 2010/0323020 A1
(43) Pub. Date: Dec. 23, 2010

(54) STABLE NANOPARTICULATE DRUG SUSPENSION

(75) Inventors: Rajeev Gokhale, Libertyville, IL (US); Kennan C. Marsh, Lake Forest, IL (US); Yi Shi, Libertyville, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/817,640

(22) Filed: Jun. 17, 2010

Related U.S. Application Data
(60) Provisional application No. 61/218,281, filed on Jun. 18, 2009.

(51) Int. Cl. A61K 31/5377; A61K 9/14; A61P 35/04; A61P 35/00; A61P 7/00

(52) U.S. Cl. 424/489; 514/235.8

(57) ABSTRACT

A liquid pharmaceutical composition comprises an aqueous medium having suspended therein a solid particulate Bc1-2 family protein inhibitory compound such as ABT-263, having a D₉₀ particle size not greater than about 3 μm; wherein the aqueous medium further comprises at least one pharmaceutically acceptable surfactant and at least one pharmaceutically acceptable basifying agent such as sodium bicarbonate in amounts that are effective together to inhibit particle size increase. The composition is suitable for oral or parenteral administration to a subject in need thereof for treatment of a disease characterized by overexpression of one or more anti-apoptotic Bc1-2 family proteins, for example cancer.

This application claims priority benefit of U.S. provisional application Ser. No. 61/218,281 filed on Jun. 18, 2009, the entire disclosure of which is incorporated herein by reference.

### FIELD OF THE INVENTION

The present invention relates to liquid suspension formulations comprising a particulate drug compound of low solubility and to processes for preparing such formulations. The invention is particularly applicable to a class of apoptosis-promoting compounds that target Bc1-2 family proteins, thus the invention further relates to methods of use of liquid suspension formulations for treating diseases characterized by overexpression of such proteins.

### BACKGROUND OF THE INVENTION

Evasion of apoptosis is a hallmark of cancer (Hanahan & Weinberg (2000) Cell 100:57-70). Cancer cells must overcome a continual bombardment by cellular stresses such as DNA damage, oncogene activation, aberrant cell cycle progression and harsh microenvironments that would cause normal cells to undergo apoptosis. One of the primary means by which cancer cells evade apoptosis is by up-regulation of anti-apoptotic proteins of the Bc1-2 family.

Compounds that occupy the BH3 binding groove of Bc1-2 proteins have been described, for example by Bruncko et al. (2007) J. Med. Chem. 50:641-662. These compounds have included N-(4-(4-((4′-chloro-(1,1′-biphenyl)-2-yl)methyl)piperazin-1-yl)benzoyl)-4-(1R)-3-(dimethylamino)-1-((phenylsulfanyl)methyl)propyl)amino)-3-nitrobenzene-sulfonamide, otherwise known as ABT-737, which has the formula:

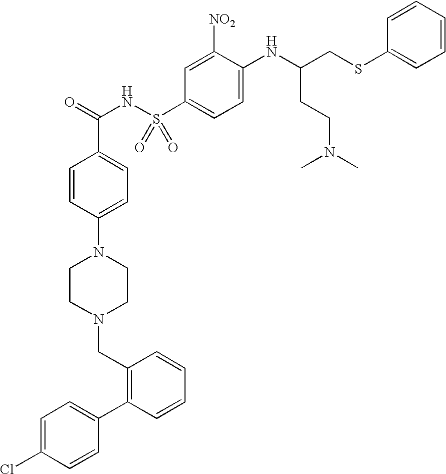

ABT-737 binds with high affinity (<1 nM) to proteins of the Bc1-2 family (specifically Bc1-2, Bc1-X_{L} and Bc1-w). It exhibits single-agent activity against small-cell lung cancer (SCLC) and lymphoid malignancies, and potentiates pro-apoptotic effects of other chemotherapeutic agents. ABT-737 and related compounds, and methods to make such compounds, are disclosed in U.S. Patent Application Publication No. 2007/0072860 of Bruncko et al.

More recently, a further series of compounds has been identified having high binding affinity to Bc1-2 family proteins. These compounds, and methods to make them, are disclosed in U.S. Patent Application Publication No. 2007/0027135 of Bruncko et al. (herein “the '135 publication”), incorporated by reference herein in its entirety, and can be seen from their formula below to be structurally related to ABT-737.

The '135 publication states that while inhibitors of Bc1-2 family proteins previously known may have either potent cellular efficacy or high systemic exposure after oral administration, they do not possess both properties. A typical measure of cellular efficacy of a compound is the concentration eliciting 50% cellular effect (EC₅₀). A typical measure of systemic exposure after oral administration of a compound is the area under the curve (AUC) resulting from graphing plasma concentration of the compound versus time from oral administration. Previously known compounds, it is stated in the '135 publication, have a low AUC/EC₅₀ ratio, meaning that they are not orally efficacious. Compounds of the above formula, by contrast, are stated to demonstrate enhanced properties with respect to cellular efficacy and systemic exposure after oral administration, resulting in a AUC/EC₅₀ ratio significantly higher than that of previously known compounds.

One compound, identified as “Example 1” in the '135 publication, is N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide, otherwise known as ABT-263. This compound has a molecular weight of 974.6 g/mol and has the formula:

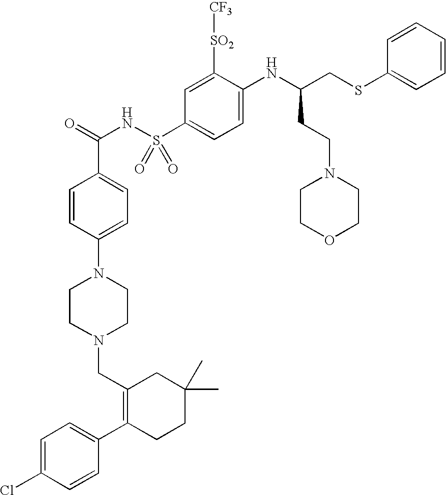

ABT-263 binds with high affinity (<1 nM) to Bc1-2 and Bc1-X_{L} and is believed to have similarly high affinity for Bc1-w. Its AUC/EC₅₀ ratio is reported in the '135 publication as 56, more than an order of magnitude greater than that reported for ABT-737 (4.5). For determination of AUC according to the '135 publication, each compound was administered to rats in a single 5 mg/kg dose by oral gavage as a 2 mg/ml solution in a vehicle of 10% DMSO (dimethyl sulfoxide) in PEG-400 (polyethylene glycol of average molecular weight about 400).

Oral bioavailability (as expressed, for example, by AUC after oral administration as a percentage of AUC after intravenous administration) is not reported in the '135 publication, but can be concluded therefrom to be substantially greater for ABT-263 than for ABT-737.

Recently, Tse et al. (2008) Cancer Res. 68(9):3421-3428, reported in supplementary data thereto that, in a dog model, oral bioavailability of an ABT-263 solution in PEG-400/DMSO was 22.4%, and that of an ABT-263 solution in 60% Phosal™ PG (phosphatidylcholine+propylene glycol), 30% PEG-400 and 10% ethanol was 47.6%.

Oxidation reactions represent an important degradation pathway of pharmaceuticals, especially when formulated in solution. Oxidation can occur by a number of pathways, including uncatalyzed autoxidation of a substrate by molecular oxygen, photolytic initiation, hemolytic thermal cleavage, and metal catalysis. Various functional groups show particular sensitivity towards oxidation. In particular, thioethers can degrade via hydrogen abstraction at the α-position to the sulfur atom or by addition of an α-peroxyl radical directly or via a one-electron transfer process, which transforms a sulfide to a sulfine, sulfone, or sulfoxide (Hovorka & Schöneich (2001) J. Pharm. Sci. 90:253-269).

The (phenylsulfanyl)methyl group possessed by compounds disclosed in the '135 publication, including ABT-263, is seen to have a thioether linkage, which is susceptible to oxidation, for example in presence of oxygen or reactive oxygen species such as superoxide, hydrogen peroxide or hydroxyl radicals. The '135 publication includes antioxidants in an extensive list of excipients said to be useful for administering the compounds disclosed therein.

However, pharmaceutical compositions that are less susceptible to oxidation of the active ingredient would be advantageous. Additionally, compositions capable of higher active ingredient loading than the solution compositions of the '135 publication or of Tse et al. (2008), supra would be advantageous.

The very low aqueous solubility of compounds of the '135 publication including ABT-263 raises challenges for the formulator, especially where there is a need to maintain acceptable oral bioavailability, which is strongly dependent on solubility in the aqueous medium of the gastrointestinal tract. Particle size reduction is commonly tried as an approach to improving bioavailability of a poorly water-soluble drug; however it is often difficult to achieve, with solid particles of any size, bioavailability comparable with that obtainable with such a drug in solution form, which can be considered to represent the ultimate in particle size reduction.

Another challenge for the formulator seeking to provide a suspension of poorly water-soluble drug particles in a liquid medium is the tendency for suspended particles, especially very small particles of around 1 μm in size or smaller, to exhibit particle size increase over time, for example through particle aggregation. Such increase in particle size can destabilize the suspension and/or lower its bioavailability. Surface modifying agents such as surfactants are widely used but not always successful. U.S. Pat. No. 7,459,283 to Wertz & Ryde describes compositions comprising nanoparticulate active agents having lysozyme as a surface stabilizer.

Möschwitzer et al. (2004) Eur. J. Pharmaceut. Biopharmaceut. 58:615-619 reported preparation of nanosuspension (defined therein as a dispersion of nanocrystals (<1,000 nm diameter) in a liquid phase) formulations of omeprazole by dispersing the drug in an aqueous medium containing 8.4% sodium bicarbonate and 1% poloxamer 188. Physical stability studies revealed moderate particle size increase over 3 days at 0° C.; the authors concluded that this size increase “of course indicates that these nanosuspensions will not possess a long-term stability of 2 years.” Chemical stability of omeprazole was reportedly greatly improved by formulating as a 50 or 100 mg/ml nanosuspension versus a 5 mg/ml aqueous solution; the authors cited possible explanations for such stability including the crystalline structure of the nanoparticles.

In this regard, ABT-263 would appear to be a poor candidate for nanosuspension formulation, as when prepared according to the '135 publication it is an amorphous solid; i.e., it lacks the crystallinity of, for example, omeprazole.

A particular type of disease for which improved therapies are needed is non-Hodgkin's lymphoma (NHL). NHL is the sixth most prevalent type of new cancer in the U.S. and occurs primarily in patients 60-70 years of age. NHL is not a single disease but a family of related diseases, which are classified on the basis of several characteristics including clinical attributes and histology.

One method of classification places different histological subtypes into two major categories based on natural history of the disease, i.e., whether the disease is indolent or aggressive. In general, indolent subtypes grow slowly and are generally incurable, whereas aggressive subtypes grow rapidly and are potentially curable. Follicular lymphomas are the most common indolent subtype, and diffuse large-cell lymphomas constitute the most common aggressive subtype. The oncoprotein Bc1-2 was originally described in non-Hodgkin's B-cell lymphoma.

Treatment of follicular lymphoma typically consists of biologically-based or combination chemotherapy. Combination therapy with rituximab, cyclophosphamide, doxorubicin, vincristine and prednisone (R-CHOP) is routinely used, as is combination therapy with rituximab, cyclophosphamide, vincristine and prednisone (RCVP). Single-agent therapy with rituximab (targeting CD20, a phosphoprotein uniformly expressed on the surface of B-cells) or fludarabine is also used. Addition of rituximab to chemotherapy regimens can provide improved response rate and increased progression-free survival.

Radioimmunotherapy agents, high-dose chemotherapy and stem cell transplants can be used to treat refractory or relapsed NHL. Currently, there is not an approved treatment regimen that produces a cure, and current guidelines recommend that patients be treated in the context of a clinical trial, even in a first-line setting.

First-line treatment of patients with aggressive large B-cell lymphoma typically consists of rituximab, cyclophosphamide, doxorubicin, vincristine and prednisone (R-CHOP), or dose-adjusted etoposide, prednisone, vincristine, cyclophosphamide, doxorubicin and rituximab (DA-EPOCH-R).

Most lymphomas respond initially to any one of these therapies, but tumors typically recur and eventually become refractory. As the number of regimens patients receive increases, the more chemotherapy-resistant the disease becomes. Average response to first-line therapy is approximately 75%, 60% to second-line, 50% to third-line, and about 35-40% to fourth-line therapy. Response rates approaching 20% with a single agent in a multiple relapsed setting are considered positive and warrant further study.

Current chemotherapeutic agents elicit their antitumor response by inducing apoptosis through a variety of mechanisms. However, many tumors ultimately become resistant to these agents. Bc1-2 and Bc1-X_{L} have been shown to confer chemotherapy resistance in short-term survival assays in vitro and, more recently, in vivo. This suggests that if improved therapies aimed at suppressing the function of Bc1-2 and Bc1-X_{L} can be developed, such chemotherapy-resistance could be successfully overcome.

Apoptosis-promoting drugs that target Bc1-2 family proteins such as Bc1-2 and Bc1-X_{L} are best administered according to a regimen that provides continual, for example daily, replenishment of the plasma concentration, to maintain the concentration in a therapeutically effective range. This can be achieved by daily parenteral, e.g., intravenous (i.v.) or intraperitoneal (i.p.) administration. However, daily parenteral administration is often not practical in a clinical setting, particularly for outpatients. To enhance clinical utility of an apoptosis-promoting agent, for example as a chemotherapeutic in cancer patients, a dosage form with acceptable oral bioavailability, but with fewer limitations than a solution formulation, would be highly desirable. Such a dosage form, and a regimen for oral administration thereof, would represent an important advance in treatment of many types of cancer, including NHL, and would more readily enable combination therapies with other chemotherapeutics.

### SUMMARY OF THE INVENTION

There is now provided a liquid pharmaceutical composition comprising an aqueous medium having suspended therein a solid particulate compound having a D₉₀ particle size not greater than about 3 μm; wherein the compound is of Formula I:

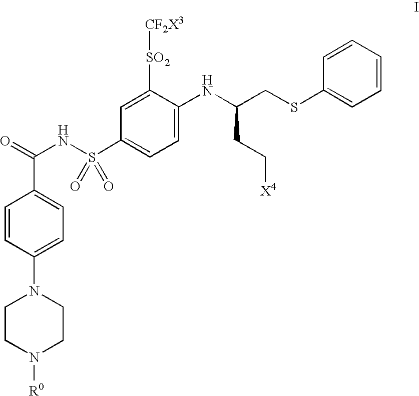

where:
X³ is chloro or fluoro; and
(1) X⁴ is azepan-1-yl, morpholin-4-yl, 1,4-oxazepan-4-yl, pyrrolidin-1-yl, —N(CH₃)₂, —N(CH₃)(CH(CH₃)₂), 7-azabicyclo[2.2.1]heptan-7-yl or 2-oxa-5-azabicyclo[2.2.1]hept-5-yl; and R⁰ is

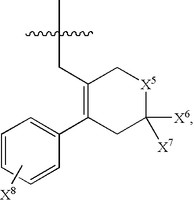

where
   X⁵ is —CH₂—, —C(CH₃)₂— or —CH₂CH₂—;
   X⁶ and X⁷ are both —H or both methyl; and
   X⁸ is fluoro, chloro, bromo or iodo;
or
(2) X⁴ is azepan-1-yl, morpholin-4-yl, pyrrolidin-1-yl, —N(CH₃)(CH(CH₃)₂) or 7-azabicyclo[2.2.1]heptan-7-yl; and R⁰ is

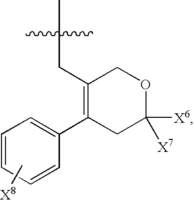

where X⁶, X⁷ and X⁸ are as above; or
(3) X⁴ is morpholin-4-yl or —N(CH₃)₂; and R⁰ is

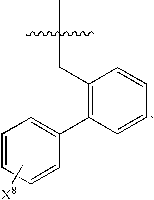

where X⁸ is as above;
   or a pharmaceutically acceptable salt, prodrug, salt of a prodrug or metabolite thereof; and wherein the aqueous medium further comprises at least one pharmaceutically acceptable surfactant and at least one pharmaceutically acceptable basifying agent in amounts that are effective together to inhibit particle size increase.

Although a composition of the invention is primarily intended for oral administration, it is generally suitable also for other routes of administration, including parenteral routes.

There is further provided a solid pharmaceutical composition comprising a compound of Formula I, or a pharmaceutically acceptable salt, prodrug, salt of a prodrug or metabolite thereof, in particulate form having a D₉₀ particle size not greater than about 3 μm; and pharmaceutically acceptable excipients including (a) at least one surfactant and at least one basifying agent and (b) at least one dispersant or bulking agent; said composition being dispersible in an aqueous medium to provide a suspension wherein the surfactant and basifying agent are in amounts that are effective together to inhibit particle size increase.

There is still further provided a process for preparing a pharmaceutical composition, comprising providing an active pharmaceutical ingredient (API) that comprises a compound of Formula I, or a pharmaceutically acceptable salt, prodrug, salt of a prodrug or metabolite thereof; wet-milling the API in presence of at least one pharmaceutically acceptable basifying agent to a D₉₀ particle size not greater than about 3 μm to provide a milled drug substance; and suspending the milled drug substance in an aqueous medium with the aid of at least one pharmaceutically acceptable surfactant; wherein the at least one basifying agent and the at least one surfactant are present in the resulting suspension in amounts that are effective together to inhibit particle size increase.

According to any of the above embodiments, the drug compound or API can be, for example, ABT-263 or a crystalline salt thereof, e.g., ABT-263 bis-hydrochloride salt (ABT-263 bis-HCl).

There is still further provided a method for treating a disease characterized by apoptotic dysfunction and/or overexpression of an anti-apoptotic Bc1-2 family protein, comprising orally administering to a subject having the disease a therapeutically effective amount of a composition as described above, e.g., such a composition comprising ABT-263 free base or ABT-263 bis-HCl. Examples of such a disease include many neoplastic diseases including cancers. A specific illustrative type of cancer that can be treated according to the present method is non-Hodgkin's lymphoma (NHL). Another specific illustrative type of cancer that can be treated according to the present method is chronic lymphocytic leukemia. Yet another specific illustrative type of cancer that can be treated according to the present method is acute lymphocytic leukemia, for example in a pediatric patient.

There is still further provided a method for maintaining in bloodstream of a human cancer patient, for example a patient having NHL, chronic lymphocytic leukemia or acute lymphocytic leukemia, a therapeutically effective plasma concentration of ABT-263 and/or one or more metabolites thereof, comprising administering to the subject a composition as described above comprising ABT-263 or a crystalline salt thereof, in a dosage amount of about 50 to about 500 mg ABT-263 free base equivalent per day, at an average dosage interval of about 3 hours to about 7 days.

Additional embodiments of the invention, including more particular aspects of those provided above, will be found in, or will be evident from, the detailed description that follows.

### BRIEF DESCRIPTION OF THE DRAWINGS

FIG. 1 is a graphical representation of ABT-263 plasma concentration over a 24-hour period following oral administration to dogs (non-fasted except where otherwise indicated) of a composition of the invention (Formulation II) and a comparative solution of ABT-263 bis-HCl in a lipid medium (Formulation C), as described in Example 3.

### DETAILED DESCRIPTION

A suspension composition in accordance with the present disclosure comprises a nanosized solid particulate drug compound. It is found that in the suspensions described herein the drug nanoparticles do not appreciably agglomerate, resulting in production of stable formulations.

Unless the context demands otherwise, the term “nanoparticle” as used herein means a particle of size (i.e., diameter in the longest dimension of the particle) not greater than about 3 μm (3,000 nm). “Nanoparticles” as recited herein therefore include not only “submicron” particles, i.e., having a size less than about 1 μm, but also “micron-sized” particles of about 1 to about 3 μm. Likewise, the adjective “nanosized” as used herein refers to nanoparticles as defined immediately above. Unless the context demands otherwise, the term “nanoparticulate” as applied to a suspension or other composition herein, and likewise the term “nanosuspension”, means having a D₉₀ particle size not greater than about 3 μm.

The D₉₀ particle size of a composition is a parameter such that 90% by volume of particles in the composition are smaller in their longest dimension than that parameter, as measured by any conventional particle size measuring technique known to those skilled in the art. Such techniques include, for example, sedimentation field flow fractionation, photon correlation spectroscopy, light scattering, and disk centrifugation. In various embodiments of the present invention, suspensions are provided having a D₉₀ particle size not greater than about 3,000 nm, not greater than about 2,000 nm, not greater than about 1,500 nm, not greater than about 1,000 nm, not greater than about 900 nm, not greater than about 800 nm, not greater than about 700 nm, not greater than about 600 nm or not greater than about 500 nm.

The D₅₀ particle size of a composition is a parameter such that 50% by volume of particles in the composition are smaller in their longest dimension than that parameter, as measured by any conventional particle size measuring technique known to those skilled in the art. D₅₀ particle size is therefore a measure of volume median particle size but is sometimes referred to as “average” or “mean” particle size. In various embodiments of the present invention, suspensions are provided having a D₅₀ particle size not greater than about 1,000 nm, not greater than about 900 nm, not greater than about 800 nm, not greater than about 700 nm, not greater than about 600 nm, not greater than about 500 nm, not greater than about 400 nm, not greater than about 350 nm or not greater than about 300 nm.

In a particular embodiment, a suspension of the invention has a D₉₀ particle size not greater than about 1,000 nm and a D₅₀ particle size not greater than about 400 nm. In another particular embodiment, a suspension of the invention has a D₉₀ particle size not greater than about 800 nm and a D₅₀ particle size not greater than about 350 nm.

The terms “low solubility” and “poorly soluble” herein refer to a solubility in water not greater than about 100 μg/ml. The present invention can be especially advantageous for drugs that are essentially insoluble in water, i.e., having a solubility of less than about 10 μg/ml. It is believed, without being bound by theory, that the advantages of nanoparticulate suspensions for such drugs arise in part not only from improved dissolution rate, which is proportional to surface area according to the well known Whitney-Noyes equation, but also from improved solubility according to the Kelvin equation. This can result in enhanced bioavailability as well as potentially reduce food effect.

It will be recognized that aqueous solubility of many compounds is pH-dependent; in the case of such compounds the solubility of interest herein is at a physiologically relevant pH, for example a pH of about 1 to about 8. Thus, in various embodiments, the drug has a solubility in water, at least at one point in a pH range from about 1 to about 8, of less than about 100 μg/ml, for example less than about 30 μg/ml, or less than about 10 μg/ml. Illustratively, ABT-263 has a solubility in water at pH 2 of less than 4 μg/ml.

In compositions of the present invention, the drug compound is a compound of Formula I as set forth above, or a pharmaceutically acceptable salt, prodrug, salt of a prodrug or metabolite thereof.

In a further embodiment, the compound has Formula I where X³ is fluoro.

In a still further embodiment, the compound has Formula I where X⁴ is morpholin-4-yl.

In a still further embodiment, the compound has Formula I where R⁰ is

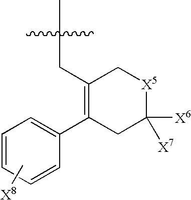

where X⁵ is —O—, —CH₂—, —C(CH₃)₂— or —CH₂CH₂—; X⁶ and X⁷ are both —H or both methyl; and X⁸ is fluoro, chloro, bromo or iodo. Illustratively according to this embodiment X⁵ can be —C(CH₃)₂— and/or each of X⁶ and X⁷ can be —H and/or X⁸ can be chloro.

In a still further embodiment, the compound has Formula I where R⁰ is

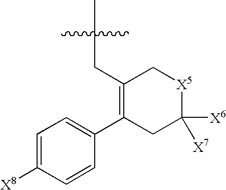

where X⁵ is —O—, —CH₂—, —C(CH₃)₂— or —CH₂CH₂—; X⁶ and X⁷ are both —H or both methyl; and X⁸ is fluoro, chloro, bromo or iodo. Illustratively according to this embodiment X⁵ can be —C(CH₃)₂— and/or each of X⁶ and X⁷ can be —H and/or X⁸ can be chloro.

In a still further embodiment, the compound has Formula I where X³ is fluoro and X⁴ is morpholin-4-yl.

In a still further embodiment, the compound has Formula I where X³ is fluoro and R⁰ is

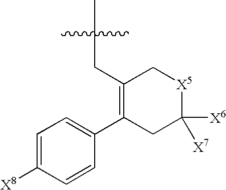

where X⁵ is —O—, —CH₂—, —C(CH₃)₂— or —CH₂CH₂—; X⁶ and X⁷ are both —H or both methyl; and X⁸ is fluoro, chloro, bromo or iodo. Illustratively according to this embodiment X⁵ can be —C(CH₃)₂— and/or each of X⁶ and X⁷ can be —H and/or X⁸ can be chloro.

In a still further embodiment, the compound has Formula I where X⁴ is morpholin-4-yl and R⁰ is

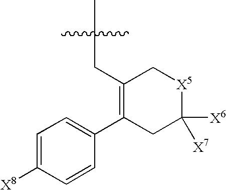

where X⁵ is —O—, —CH₂—, —C(CH₃)₂— or —CH₂CH₂—; X⁶ and X⁷ are both —H or both methyl; and X⁸ is fluoro, chloro, bromo or iodo. Illustratively according to this embodiment X⁵ can be —C(CH₃)₂— and/or each of X⁶ and X⁷ can be —H and/or X⁸ can be chloro.

In a still further embodiment, the compound has Formula I where X³ is fluoro, X⁴ is morpholin-4-yl and R⁰ is

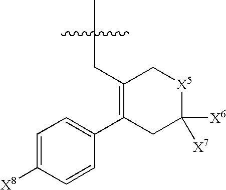

where X⁵ is —O—, —CH₂—, —C(CH₃)₂— or —CH₂CH₂—; X⁶ and X⁷ are both —H or both methyl; and X⁸ is fluoro, chloro, bromo or iodo. Illustratively according to this embodiment X⁵ can be —C(CH₃)₂— and/or each of X⁶ and X⁷ can be —H and/or X⁸ can be chloro.

Compounds of Formula I may contain asymmetrically substituted carbon atoms in the R- or S-configuration; such compounds can be present as racemates or in an excess of one configuration over the other, for example in an enantiomeric ratio of at least about 85:15. The compound can be substantially enantiomerically pure, for example having an enantiomeric ratio of at least about 95:5, or in some cases at least about 98:2 or at least about 99:1.

Compounds of Formula I may alternatively or additionally contain carbon-carbon double bonds or carbon-nitrogen double bonds in the Z- or E-configuration, the term “Z” denoting a configuration wherein the larger substituents are on the same side of such a double bond and the term “E” denoting a configuration wherein the larger substituents are on opposite sides of the double bond. The compound can alternatively be present as a mixture of Z- and E-isomers.

Compounds of Formula I may alternatively or additionally exist as tautomers or equilibrium mixtures thereof wherein a proton shifts from one atom to another. Examples of tautomers illustratively include keto-enol, phenol-keto, oxime-nitroso, nitro-aci, imine-enamine and the like.

In some embodiments, a compound of Formula I is present in the nanoparticulate suspension in its parent-compound form, alone or together with a salt or prodrug form of the compound.

Compounds of Formula I may form acid addition salts, basic addition salts or zwitterions. Salts of compounds of Formula I can be prepared during isolation or following purification of the compounds. Acid addition salts are those derived from reaction of a compound of Formula I with an acid. For example, salts including the acetate, adipate, alginate, bicarbonate, citrate, aspartate, benzoate, benzenesulfonate (besylate), bisulfate, butyrate, camphorate, camphorsulfonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetate, trifluoroacetate, para-toluenesulfonate and undecanoate salts of a compound of Formula I can be used in a composition of the invention. Basic addition salts, including those derived from reaction of a compound with the bicarbonate, carbonate, hydroxide or phosphate of cations such as lithium, sodium, potassium, calcium and magnesium, can likewise be used.

A compound of Formula I typically has more than one protonatable nitrogen atom and is consequently capable of forming acid addition salts with more than one, for example about 1.2 to about 2, about 1.5 to about 2 or about 1.8 to about 2, equivalents of acid per equivalent of the compound.

ABT-263 can likewise form acid addition salts, basic addition salts or zwitterions. Salts of ABT-263 can be prepared during isolation or following purification of the compound. Acid addition salts derived from reaction of ABT-263 with an acid include those listed above. Basic addition salts including those listed above can likewise be used. ABT-263 has at least two protonatable nitrogen atoms and is consequently capable of forming acid addition salts with more than one, for example about 1.2 to about 2, about 1.5 to about 2 or about 1.8 to about 2, equivalents of acid per equivalent of the compound.

Illustratively in the case of ABT-263, bis-salts can be formed including, for example, bis-hydrochloride (bis-HCl) and bis-hydrobromide (bis-HBr) salts.

For example, ABT-263 bis-HCl, which has a molecular weight of 1047.5 g/mol and is represented by the formula

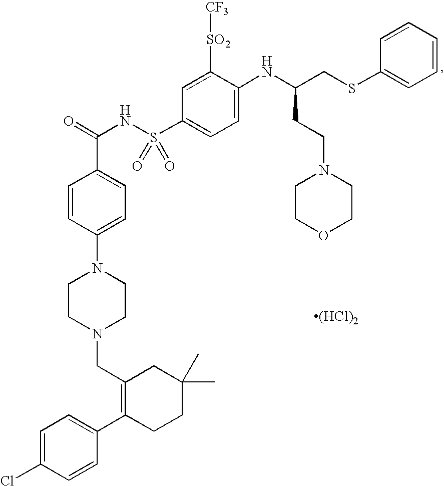

can be prepared by a variety of processes, for example a process that can be outlined as follows.

ABT-263 free base is prepared, illustratively as described in Example 1 of above-cited U.S. Patent Application Publication No. 2007/0027135, the entire disclosure of which is incorporated by reference herein. A suitable weight of ABT-263 free base is dissolved in ethyl acetate. A solution of hydrochloric acid in ethanol (for example about 4.3 kg HCl in 80 g ethanol) is added to the ABT-263 solution in an amount providing at least 2 mol HCl per mol ABT-263 and sufficient ethanol (at least about 20 vol) for crystallization of the resulting ABT-263 bis-HCl salt. The solution is heated to about 45° C. with stirring and seeds are added as a slurry in ethanol. After about 6 hours, the resulting slurry is cooled to about 20° C. over about 1 hour and is mixed at that temperature for about 36 hours. The slurry is filtered to recover a crystalline solid, which is an ethanol solvate of ABT-263 bis-HCl. Drying of this solid under vacuum and nitrogen with mild agitation for about 8 days yields white desolvated ABT-263 bis-HCl crystals. This material is suitable as an API for preparation of a composition of the present invention.

The term “free base” is used for convenience herein to refer to the parent compound, while recognizing that the parent compound is, strictly speaking, zwitterionic and thus does not always behave as a true base.

Compounds of Formula I, and methods of preparation of such compounds, are disclosed in above-cited U.S. Patent Application Publication No. 2007/0027135 and/or in above-cited U.S. Patent Application Publication No. 2007/0072860, each of which is incorporated herein by reference in its entirety. Terms for substituents used herein are defined exactly as in those publications.

Compounds of Formula I having —NH, —C(O)OH, —OH or —SH moieties may have attached thereto prodrug-forming moieties which can be removed by metabolic processes in vivo to release the parent compound having free —NH, —C(O)OH, —OH or —SH moieties. Salts of prodrugs can also be used.

Without being bound by theory, it is believed that the therapeutic efficacy of compounds of Formula I is due at least in part to their ability to bind to a Bc1-2 family protein such as Bc1-2, Bc1-X_{L} or Bc1-w in a way that inhibits the anti-apoptotic action of the protein, for example by occupying the BH3 binding groove of the protein. It will generally be found desirable to select a compound having high binding affinity for a Bc1-2 family protein, for example a Kᵢ not greater than about 5 nM, preferably not greater than about 1 nM.

The nanoparticulate suspension comprises a compound of Formula I or a salt, prodrug, salt of a prodrug or metabolite thereof as a discrete solid-state phase that can be crystalline, semi-crystalline or amorphous. In the case of ABT-263, the free base form of which, as prepared according to the '135 publication, is an amorphous or glassy solid, it is generally preferred to use a crystalline salt form of the drug, such as for example ABT-263 bis-HCl, in preparing the nanosuspension. However, upon suspension of the salt in presence of a basifying agent such as sodium bicarbonate, some conversion of salt to free base can occur, resulting in the solid-state phase becoming at least partly amorphous. Accordingly, in one embodiment, the nanosuspension comprises ABT-263 free base, ABT-263 bis-HCl or a combination thereof. Despite the likelihood that the drug particles in an ABT-263 nanosuspension are at least partly amorphous, a remarkably high degree of physical stability has been observed in such a nanosuspension, as illustrated in Example 2 below.

The present inventors have found that nanoparticulate suspensions as described herein offer not only the advantage of physical stability providing acceptable product shelf life, but also the robustness of manufacturing process that is desirable for a commercial product.

A compound of Formula I or a salt, prodrug, salt of a prodrug or metabolite thereof is present in a nanoparticulate suspension of the invention in an amount that can be therapeutically effective when the composition is administered to a subject in need thereof according to an appropriate regimen. Dosage amounts are expressed herein as parent-compound-equivalent (free base equivalent) amounts unless the context requires otherwise. Typically, a unit dose (the amount administered at a single time), which can be administered at an appropriate frequency, e.g., twice daily to once weekly, is about 10 to about 1,000 mg, depending on the compound in question. Where frequency of administration is once daily (q.d.), unit dose and daily dose are the same. Illustratively, for example where the drug is ABT-263, the unit dose is typically about 25 to about 1,000 mg, more typically about 50 to about 500 mg, for example about 50, about 100, about 150, about 200, about 250, about 300, about 350, about 400, about 450 or about 500 mg, free base equivalent. Where the dosage form comprises a capsule shell enclosing the nanoparticulate composition in suspension or solid form, or is a tablet comprising the nanoparticulate composition in solid form, a unit dose can be deliverable in a single capsule or tablet or a plurality of capsules or tablets, most typically 1 to about 10 capsules or tablets.

The higher the unit dose, the more desirable it becomes to select a suspension having a relatively high concentration of the drug therein. Typically, the concentration of drug in the suspension is at least about 10 mg/ml, e.g., about 10 to about 500 mg/ml, but lower and higher concentrations can be acceptable or achievable in specific cases. Illustratively, for example where the drug is ABT-263, the drug concentration in various embodiments is at least about 10 mg/ml, e.g., about 10 to about 400 mg/ml, or at least about 20 mg/ml, e.g., about 20 to about 200 mg/ml, for example about 20, about 25, about 30, about 40, about 50, about 75, about 100, about 125, about 150 or about 200 mg/ml, by free base equivalent weight.

Compositions of the present invention have good storage-stability properties. In particular, they are physically stable, at least in that they do not have an unacceptable tendency to undergo particle size increase over time, for example through particle agglomeration. Particle agglomeration is a common problem in nanoparticulate suspensions. Surface modifying agents such as surfactants are important in reducing the tendency of nanoparticles to agglomerate; the at least one surfactant present in a composition of the present invention is believed, without being bound by theory, to help in this regard.

A “basifying agent” herein is any agent that raises the pH of the suspension medium. Any pharmaceutically acceptable basifying agent can be used, including without limitation hydroxides and bicarbonates of alkali metals such as sodium and potassium. The invention is illustrated herein with particular reference to sodium bicarbonate, but it will be recognized that other basifying agents can be substituted for sodium bicarbonate if desired.

Amount of sodium bicarbonate useful in a composition of the invention is not narrowly critical, and one of ordinary skill in the art can readily optimize the amount for any particular composition, for example by routine storage-stability testing. In general, good results can be obtained with sodium bicarbonate in an amount of about 20 to about 200 mg/ml, for example about 40 to about 160 mg/ml.

The choice and amount of surfactant is likewise not narrowly critical, and is likely to depend to some extent on the particular drug compound to be formulated and the drug loading desired. Non-limiting examples of surfactants include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers), for example poloxamer 188 and poloxamer 237; polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters, for example sorbitan monolaurate, sorbitan monooleate, sorbitan monopalmitate and sorbitan monostearate; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; α-tocopheryl polyethylene glycol succinate (TPGS); tyloxapol; and the like. In one embodiment, the at least one surfactant is a poloxamer or mixture of poloxamers. Poloxamer 188 is a specific example. One or more surfactants typically constitute in total about 10 to about 100 mg/ml. In the case of poloxamer 188, an illustratively suitable amount is about 10 to about 100 mg/ml, for example about 15 to about 60 mg/ml.

The aqueous medium of the suspension can take the form of water, an aqueous injectable fluid such as saline (e.g., phosphate-buffered saline or PBS) or an imbibable liquid such as fruit juice or a carbonated beverage. In one embodiment the nanoparticulate drug compound, the at least one surfactant and at least one basifying agent (and optionally additional ingredients) are prepared as a dry powder mix for reconstitution with a suitable aqueous medium to form a suspension composition of the invention shortly before use. Such a reconstitutable powder should contain, in addition to the ingredients recited above, at least one pharmaceutically acceptable dispersant or bulking agent, typically a water-soluble material such as a sugar, e.g., dextrose, mannitol or dextran; a phosphate salt, e.g., sodium or potassium phosphate; an organic acid, e.g., citric acid or tartaric acid, or a salt thereof; or a mixture of such materials. A dry powder mix can alternatively be administered to a subject for resuspension of the nanoparticles in the gastrointestinal fluid; for such administration the powder mix can if desired be formed into a tablet or filled into a capsule.

In the case of a compound of Formula I, it is desirable to provide a formulation that is not only physically stable but also chemically stable. More particularly, such a formulation should not exhibit an unacceptable degree of oxidative degradation of the compound of Formula I, for example at the thioether linkage of the (phenylsulfanyl)methyl group thereof.

In this regard, a composition of the present invention containing a compound of Formula I such as ABT-263 free base, ABT-263 bis-HCl or a combination thereof possesses a significant advantage over solution compositions of ABT-263 previously disclosed in the art, for example in the '135 publication or in Tse et al. (2008), supra. The solid-state form (whether crystalline, semi-crystalline or amorphous) of ABT-263 present in a nanosuspension as provided herein is believed to be significantly more resistant to oxidative degradation than ABT-263 in solution.

However, if desired, any remaining tendency for oxidative degradation can be further reduced by inclusion of a suitable antioxidant in the suspension composition.

An “antioxidant” or compound having “antioxidant” properties is a chemical compound that prevents, inhibits, reduces or retards oxidation of another chemical or itself. Antioxidants can improve stability and shelf-life of a lipid formulation as described herein by, for example, preventing, inhibiting, reducing or retarding oxidation of the compound of Formula I in the formulation.

Enhancement of stability or shelf-life can be evaluated, for example, by monitoring rate of appearance or build-up of sulfoxides in the formulation. Sulfoxides in total can be monitored by repeated sampling and analysis; alternatively samples can be analyzed more specifically for the sulfoxide degradation product of the compound of Formula I, i.e., the compound having the formula

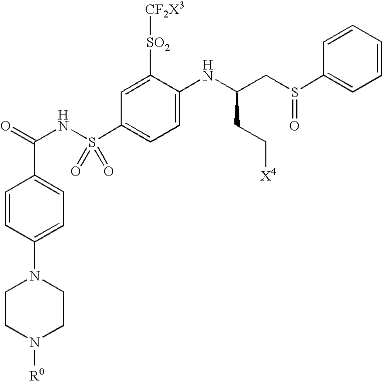

where X³, X⁴ and R⁰ are as indicated above; or the sulfoxide degradation product of ABT-263, having the formula

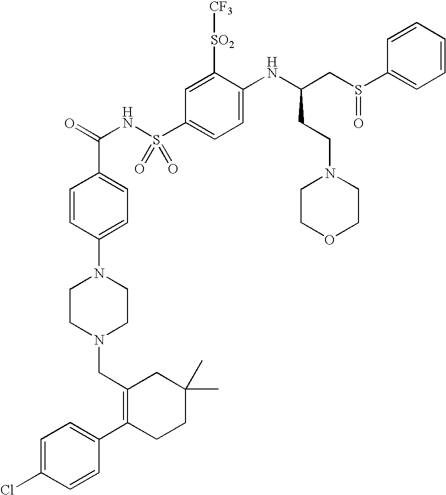

Reference herein to the sulfoxide degradation product will be understood to include both diastereomers at the sulfur atom stereocenter in the sulfoxide group.

An “antioxidant effective amount” of an antioxidant herein is an amount that provides
(a) a substantial reduction (for example a reduction of at least about 25%, at least about 50%, at least about 75%, at least about 80%, at least about 85% or at least about 90%) in the formation or accumulation of a degradation product, for example the sulfoxide degradation product above, and/or
(b) a substantial increase (for example at least about 30, at least about 60, at least about 90 or at least about 180 days) in the time taken for the degradation product to reach a threshold level,
   in a formulation containing the antioxidant, by comparison with an otherwise similar formulation containing no antioxidant. A storage-stability study to determine degree of (a) reduction in formation or accumulation of the degradation product or (b) increase in time taken for a degradation product to reach a threshold level in the formulation can be conducted at any appropriate temperature or range of temperatures. Illustratively, a study at about 5° C. can be indicative of storage stability under refrigerated conditions, a study at about 20-25° C. can be indicative of storage stability under typical ambient conditions, and a study at about 30° C. or higher temperature can be useful in an accelerated-aging study. Any appropriate threshold level of the degradation product can be selected as an end-point, for example in the range from about 0.2% to about 2% of the initial amount of the compound of Formula I present.

In various illustrative embodiments, the antioxidant is included in an amount effective to hold oxidative degradation of the drug

(a) below about 1% for at least about 3 months;

(b) below about 1% for at least about 6 months;

(c) below about 1% for at least about 1 year;

(d) below about 0.5% for at least about 3 months;

(e) below about 0.5% for at least about 6 months; or

(f) below about 0.5% for at least about 1 year;

in the formulation when stored under ambient conditions (e.g., about 20-25° C.) in a sealed container opaque to ultraviolet light, as measured for example by amount of the sulfoxide degradation product present at the end of the recited storage period.

Antioxidants used in pharmaceutical compositions are most typically agents that inhibit generation of oxidative species such as triplet or singlet oxygen, superoxides, peroxide and free hydroxyl radicals, or agents that scavenge such oxidative species as they are generated. Examples of commonly used antioxidants of these classes include butylated hydroxyanisole (BHA), butylated hydroxytoluene (BHT), retinyl palmitate, tocopherol, propyl gallate, ascorbic acid and ascorbyl palmitate. Antioxidants useful herein, however, are heavier-chalcogen antioxidants that are believed, without being bound by theory, to function primarily as competitive substrates, i.e., as “sacrificial” antioxidants, which are preferentially attacked by oxidative species thereby protecting the drug from excessive degradation.

In some embodiments, the HCA comprises one or more antioxidant compounds of Formula II:

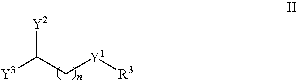

where

n is 0, 1 or 2;

Y¹ is S or Se;

Y² is NHR¹, OH or H, where R^{l} is alkyl or alkylcarbonyl;

Y³ is COOR² or CH₂OH, where R² is H or alkyl; and

R³ is H or alkyl;

where alkyl groups are independently optionally substituted with one of more substituents independently selected from the group consisting of carboxyl, alkylcarbonyl, alkoxycarbonyl, amino and alkylcarbonylamino; a pharmaceutically acceptable salt thereof; or, where Y¹ is S and R³ is H, an —S—S— dimer thereof or pharmaceutically acceptable salt of such dimer.

In other embodiments, the HCA is an antioxidant compound of Formula III:

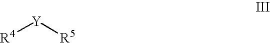

where
Y is S, Se or S—S; and
R⁴ and R⁵ are independently selected from H, alkyl and (CH₂)ₙR⁶ where n is 0-10 and R⁶ is arylcarbonyl, alkylcarbonyl, alkoxycarbonyl, carboxyl or CHR⁷R⁸-substituted alkyl, where R⁷ and R⁸ are independently CO₂R⁹, CH₂OH, hydrogen or NHR¹⁰, where R⁹ is H, alkyl, substituted alkyl or arylalkyl and R¹⁰ is hydrogen, alkyl, alkylcarbonyl or alkoxycarbonyl.

An “alkyl” substituent or an “alkyl” or “alkoxy” group forming part of a substituent according to Formula II or Formula III is one having 1 to about 18 carbon atoms and can consist of a straight or branched chain.

An “aryl” group forming part of a substituent according to Formula III is a phenyl group, unsubstituted or substituted with one or more hydroxy, alkoxy or alkyl groups.

In some embodiments, R^{l} in Formula II is C₁₋₄ alkyl (e.g., methyl or ethyl) or (C₁₋₄ alkyl)carbonyl (e.g., acetyl).

In some embodiments, R² in Formula II is H or C₁₋₁₈ alkyl, for example methyl, ethyl, propyl (e.g., n-propyl or isopropyl), butyl (e.g., n-butyl, isobutyl or t-butyl), octyl (e.g., n-octyl or 2-ethylhexyl), dodecyl (e.g., lauryl), tridecyl, tetradecyl, hexadecyl or octadecyl (e.g., stearyl).

R³ is typically H or C₁₋₄ alkyl (e.g., methyl or ethyl).

The HCA can be, for example, a natural or synthetic amino acid or a derivative thereof such as an alkyl ester or N-acyl derivative, or a salt of such amino acid or derivative. Where the amino acid or derivative thereof is derived from a natural source it is typically in the L-configuration; however it is understood that D-isomers and D,L-isomer mixtures can be substituted if necessary.

Non-limiting examples of HCAs useful herein include β-alkylmercaptoketones, cysteine, cystine, homocysteine, methionine, thiodiglycolic acid, thiodipropionic acid, thioglycerol, selenocysteine, selenomethionine and salts, esters, amides and thioethers thereof; and combinations thereof. More particularly, one or more HCAs can be selected from N-acetylcysteine, N-acetylcysteine butyl ester, N-acetylcysteine dodecyl ester, N-acetyl-cysteine ethyl ester, N-acetylcysteine methyl ester, N-acetylcysteine octyl ester, N-acetyl-cysteine propyl ester, N-acetylcysteine stearyl ester, N-acetylcysteine tetradecyl ester, N-acetylcysteine tridecyl ester, N-acetylmethionine, N-acetylmethionine butyl ester, N-acetylmethionine dodecyl ester, N-acetylmethionine ethyl ester, N-acetylmethionine methyl ester, N-acetylmethionine octyl ester, N-acetylmethionine propyl ester, N-acetylmethionine stearyl ester, N-acetylmethionine tetradecyl ester, N-acetylmethionine tridecyl ester, N-acetyl-selenocysteine, N-acetylselenocysteine butyl ester, N-acetylselenocysteine dodecyl ester, N-acetylselenocysteine ethyl ester, N-acetylselenocysteine methyl ester, N-acetylseleno-cysteine octyl ester, N-acetylselenocysteine propyl ester, N-acetylselenocysteine stearyl ester, N-acetylselenocysteine tetradecyl ester, N-acetylselenocysteine tridecyl ester, N-acetylseleno-methionine, N-acetylselenomethionine butyl ester, N-acetylselenomethionine dodecyl ester, N-acetylselenomethionine ethyl ester, N-acetylselenomethionine methyl ester, N-acetyl-selenomethionine octyl ester, N-acetylselenomethionine propyl ester, N-acetylseleno-methionine stearyl ester, N-acetylselenomethionine tetradecyl ester, N-acetylseleno-methionine tridecyl ester, cysteine, cysteine butyl ester, cysteine dodecyl ester, cysteine ethyl ester, cysteine methyl ester, cysteine octyl ester, cysteine propyl ester, cysteine stearyl ester, cysteine tetradecyl ester, cysteine tridecyl ester, cystine, cystine dibutyl ester, cystine di(dodecyl)ester, cystine diethyl ester, cystine dimethyl ester, cystine dioctyl ester, cystine dipropyl ester, cystine distearyl ester, cystine di(tetradecyl)ester, cystine di(tridecyl)ester, N,N-diacetylcystine, N,N-diacetylcystine dibutyl ester, N,N-diacetylcystine diethyl ester, N,N-diacetylcystine di(dodecyl)ester, N,N-diacetylcystine dimethyl ester, N,N-diacetylcystine dioctyl ester, N,N-diacetylcystine dipropyl ester, N,N-diacetylcystine distearyl ester, N,N-diacetylcystine di(tetradecyl)ester, N,N-diacetylcystine di(tridecyl)ester, dibutyl thiodiglycolate, dibutyl thiodipropionate, di(dodecyl)thiodiglycolate, di(dodecyl) thiodipropionate, diethyl thiodiglycolate, diethyl thiodipropionate, dimethyl thiodiglycolate, dimethyl thiodipropionate, dioctyl thiodiglycolate, dioctyl thiodipropionate, dipropyl thiodiglycolate, dipropyl thiodipropionate, distearyl thiodiglycolate, distearyl thiodipropionate, di(tetradecyl)thiodiglycolate, di(tetradecyl)thiodipropionate, homocysteine, homocysteine butyl ester, homocysteine dodecyl ester, homocysteine ethyl ester, homocysteine methyl ester, homocysteine octyl ester, homocysteine propyl ester, homocysteine stearyl ester, homocysteine tetradecyl ester, homocysteine tridecyl ester, methionine, methionine butyl ester, methionine dodecyl ester, methionine ethyl ester, methionine methyl ester, methionine octyl ester, methionine propyl ester, methionine stearyl ester, methionine tetradecyl ester, methionine tridecyl ester, S-methylcysteine, S-methyl-cysteine butyl ester, S-methylcysteine dodecyl ester, S-methylcysteine ethyl ester, S-methyl-cysteine methyl ester, S-methylcysteine octyl ester, S-methylcysteine propyl ester, S-methyl-cysteine stearyl ester, S-methylcysteine tetradecyl ester, S-methylcysteine tridecyl ester, selenocysteine, selenocysteine butyl ester, selenocysteine dodecyl ester, selenocysteine ethyl ester, selenocysteine methyl ester, selenocysteine octyl ester, selenocysteine propyl ester, selenocysteine stearyl ester, selenocysteine tetradecyl ester, selenocysteine tridecyl ester, selenomethionine, selenomethionine butyl ester, selenomethionine dodecyl ester, seleno-methionine ethyl ester, selenomethionine methyl ester, selenomethionine octyl ester, seleno-methionine propyl ester, selenomethionine stearyl ester, selenomethionine tetradecyl ester, selenomethionine tridecyl ester, thiodiglycolic acid, thiodipropionic acid, thioglycerol, isomers and mixtures of isomers thereof, and salts thereof.

Salts of HCA compounds can be acid addition salts such as the acetate, adipate, alginate, bicarbonate, citrate, aspartate, benzoate, benzenesulfonate (besylate), bisulfate, butyrate, camphorate, camphorsulfonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetate, trifluoroacetate, para-toluenesulfonate and undecanoate salts. In a particular embodiment, the hydrochloride salt of one of the compounds individually mentioned above is present in the composition in an antioxidant effective amount.

Without being bound by theory, it is generally believed that heavier-chalcogen antioxidants such as those exemplified above protect the active compound by being themselves more readily oxidizable and, therefore, being oxidized preferentially over the drug compound. In general, for this mode of operation to provide an acceptable degree of protection for the drug compound, the antioxidant must be present in a substantial amount, for example in a molar ratio to the drug compound of at least about 1:10. In some embodiments, the molar ratio of antioxidant to the drug compound is about 1:10 to about 2:1, for example about 1:5 to about 1.5:1. Best results will sometimes be obtained when the molar ratio is approximately 1:1, i.e., about 8:10 to about 10:8.

Another class of sulfur-containing antioxidants, namely inorganic antioxidants of the sulfite, bisulfite, metabisulfite and thiosulfate classes, can be useful in compositions of the present invention. These antioxidants are used in aqueous solution. Sodium and potassium salts of sulfites, bisulfites, metabisulfites and thiosulfates are useful antioxidants according to the present embodiment; more particularly sodium and potassium metabisulfites. Such sulfur-containing antioxidants can be effective at much lower concentrations than those providing molar equivalence to the concentration of drug compound, for example at a molar ratio to the drug compound as low as 1:20 or even lower.

To further minimize sulfoxide formation, a chelating agent such as EDTA or a salt thereof (e.g., disodium EDTA or calcium disodium EDTA) is optionally added, for example in an amount of about 0.002% to about 0.02% by weight of the composition. Chelating agents sequester metal ions that can promote oxidative degradation.

Sulfoxide formation can be further minimized by selecting formulation ingredients having low peroxide value. Peroxide value is a well established property of pharmaceutical excipients and is generally expressed (as herein) in units corresponding to milliequivalents of peroxides per kilogram of excipient (meq/kg). Some excipients inherently have low peroxide value, but others, for example those having unsaturated fatty acid such as oleyl moieties and/or polyoxyethylene chains, can be sources of peroxides.

Other optional ingredients of the suspension composition include buffers, coloring agents, flavoring agents, preservatives, sweeteners, tonicifying agents and combinations thereof.

In an embodiment of the invention, a process for preparing a pharmaceutical composition comprises providing an active pharmaceutical ingredient (API) that comprises a compound of Formula I, or a pharmaceutically acceptable salt, prodrug, salt of a prodrug or metabolite thereof, for example ABT-263 or a crystalline salt thereof; wet-milling the API in presence of at least one basifying agent, such as sodium bicarbonate, to a D₉₀ particle size not greater than about 3 μm to provide a milled drug substance; and suspending the milled drug substance in an aqueous medium with the aid of at least one surfactant; wherein the at least one basifying agent and the at least one surfactant are present in the resulting suspension in amounts that are effective together to inhibit particle size increase.

Any suitable wet-milling process can be used. A particular wet-milling process that has been found useful is high-pressure homogenization as illustratively described in Example 1 below.

The present invention is not limited to compositions prepared by any process described herein; however, a composition prepared by the above process is a particular embodiment of the invention.

In one embodiment, the process further comprises adding at least one pharmaceutically acceptable dispersant or bulking agent to the suspension, drying (for example freeze-drying or lyophilizing, or alternatively spray-drying) the suspension to provide a reconstitutable dry powder, and optionally forming the powder into a tablet (for example by molding or compression) or filling the powder into a capsule, to prepare a unit dosage form.

In addition to the stabilizing benefits of sodium bicarbonate, it is found that in presence of sodium bicarbonate wet-milling to smaller particle sizes, for example to a D₉₀ particle size not greater than about 700 nm, is possible. Without sodium bicarbonate, as illustratively shown in Example 2 hereinbelow, using the same processing parameters, D₉₀ particle size can not be reduced below about 1,000 nm. The wet-milling method used in the present process has the advantage, by comparison with dry-milling, that it reduces exposure of the API to high temperature and thereby reduces risk of thermal decomposition of the API. In one embodiment, processing temperature is controlled, for example within about 1 to about 5 degrees of a target temperature of about 5° C. to about 30° C. This can be achieved by conventional means, such as by running the formulation through a heat exchanger immersed in a chilled water bath.

The composition can be prepared for wet-milling at its final concentration, or it can be prepared at higher concentration and diluted to a desired concentration after wet-milling. The at least one surfactant and, if desired, optional additional ingredients, can be added before or after wet-milling.

A composition of the invention is typically “orally deliverable”, i.e., adapted for oral administration; however, such a composition can be useful for delivery of the drug to a subject in need thereof by other routes of administration, including without limitation parenteral, sublingual, buccal, intranasal, pulmonary, topical, transdermal, intradermal, ocular, otic, rectal, vaginal, intragastric, intracranial, intrasynovial and intra-articular routes. In particular embodiments the composition is adapted for oral and/or parenteral administration.

The terms “oral administration” and “orally administered” herein refer to administration to a subject per os (p.o.), that is, administration wherein the composition is immediately swallowed, for example with the aid of a suitable volume of water or other potable liquid. “Oral administration” is distinguished herein from intraoral administration, e.g., sublingual or buccal administration or topical administration to intraoral tissues such as periodontal tissues, that does not involve immediate swallowing of the composition.

It has unexpectedly been found that a nanoparticulate ABT-263 bis-HCl suspension of the invention provides enhanced bioabsorption by comparison with a standard solution of the drug, e.g., a solution in a carrier consisting of 10% DMSO in PEG-400 as disclosed in the '135 publication, when administered orally. Indeed bioabsorption is found to be comparable with that obtained with a lipid solution formulation of ABT-263 bis-HCl (herein “Formulation C”) presently in clinical trials (see Example 3 below). Enhanced bioabsorption can be evidenced, for example, by a pharmacokinetic (PK) profile having one or more of a higher Cₘₐₓ or an increased bioavailability as measured by AUC, for example AUC₀₋₂₄ or AUC_{0-∞}. Illustratively, bioavailability can be expressed as a percentage, for example using the parameter F, which computes AUC for oral delivery of a test composition as a percentage of AUC for intravenous (i.v.) delivery of the drug in a suitable solvent, taking into account any difference between oral and i.v. doses.

Bioavailability can be determined by PK studies in humans or in any suitable model species. For present purposes, a dog model, as illustratively described in Example 3 below, is generally suitable. In various illustrative embodiments, where the drug is a crystalline salt of ABT-263 such as ABT-263 bis-HCl, compositions of the invention exhibit oral bioavailability of at least about 15%, at least about 20% or at least about 25%, up to or exceeding about 50%, in a dog model, when administered as a single dose of about 2.5 to about 10 mg/kg to fasting or non-fasting animals.

Compositions embraced herein, including compositions described generally or with specificity herein, are useful for orally delivering a drug that is a compound of Formula I or a pharmaceutically acceptable salt, prodrug, salt of a prodrug or metabolite thereof to a subject. Accordingly, a method of the invention for delivering such a drug to a subject comprises orally administering a composition as described above.

The subject can be human or non-human (e.g., a farm, zoo, work or companion animal, or a laboratory animal used as a model) but in an important embodiment the subject is a human patient in need of the drug, for example to treat a disease characterized by apoptotic dysfunction and/or overexpression of an anti-apoptotic Bc1-2 family protein. A human subject can be male or female and of any age, but is typically an adult.

The composition is normally administered in an amount providing a therapeutically effective daily dose of the drug. The term “daily dose” herein means the amount of drug administered per day, regardless of the frequency of administration. For example, if the subject receives a unit dose of 150 mg twice daily, the daily dose is 300 mg. Use of the term “daily dose” will be understood not to imply that the specified dosage amount is necessarily administered once daily. However, in a particular embodiment the dosing frequency is once daily (q.d.), and the daily dose and unit dose are in this embodiment the same thing.

What constitutes a therapeutically effective dose depends on the particular compound, the subject (including species and body weight of the subject), the disease (e.g., the particular type of cancer) to be treated, the stage and/or severity of the disease, the individual subject's tolerance of the compound, whether the compound is administered in monotherapy or in combination with one or more other drugs, e.g., other chemotherapeutics for treatment of cancer, and other factors. Thus the daily dose can vary within wide margins, for example from about 10 to about 1,000 mg. Greater or lesser daily doses can be appropriate in specific situations. It will be understood that recitation herein of a “therapeutically effective” dose herein does not necessarily require that the drug be therapeutically effective if only a single such dose is administered; typically therapeutic efficacy depends on the composition being administered repeatedly according to a regimen involving appropriate frequency and duration of administration. It is strongly preferred that, while the daily dose selected is sufficient to provide benefit in terms of treating the cancer, it should not be sufficient to provoke an adverse side-effect to an unacceptable or intolerable degree. A suitable therapeutically effective dose can be selected by the physician of ordinary skill without undue experimentation based on the disclosure herein and on art cited herein, taking into account factors such as those mentioned above. The physician may, for example, start a cancer patient on a course of therapy with a relatively low daily dose and titrate the dose upwards over a period of days or weeks, to reduce risk of adverse side-effects.

Illustratively, suitable doses of ABT-263 are generally about 25 to about 1,000 mg/day, more typically about 50 to about 500 mg/day or about 200 to about 400 mg/day, for example about 50, about 100, about 150, about 200, about 250, about 300, about 350, about 400, about 450 or about 500 mg/day, administered at an average dosage interval of about 3 hours to about 7 days, for example about 8 hours to about 3 days, or about 12 hours to about 2 days. In most cases a once-daily (q.d.) administration regimen is suitable.

An “average dosage interval” herein is defined as a span of time, for example one day or one week, divided by the number of unit doses administered over that span of time. For example, where a drug is administered three times a day, around 8 am, around noon and around 6 pm, the average dosage interval is 8 hours (a 24-hour time span divided by 3). If the drug is formulated as a discrete dosage form such as a tablet or capsule, a plurality (e.g., 2 to about 10) of dosage forms administered at one time is considered a unit dose for the purpose of defining the average dosage interval.

Where the drug compound is ABT-263, for example in the form of ABT-263 bis-HCl, a daily dosage amount and dosage interval can, in some embodiments, be selected to maintain a plasma concentration of ABT-263 in a range of about 0.5 to about 10 μg/ml. Thus, during a course of ABT-263 therapy according to such embodiments, the steady-state peak plasma concentration (Cₘₐₓ) should in general not exceed about 10 μg/ml, and the steady-state trough plasma concentration (Cₘᵢₙ) should in general not fall below about 0.5 μg/ml. It will further be found desirable to select, within the ranges provided above, a daily dosage amount and average dosage interval effective to provide a Cₘₐₓ/Cₘᵢₙ ratio not greater than about 5, for example not greater than about 3, at steady-state. It will be understood that longer dosage intervals will tend to result in greater Cₘₐₓ/Cₘᵢₙ ratios. Illustratively, at steady-state, an ABT-263 Cₘₐₓ of about 3 to about 8 μg/ml and Cₘᵢₙ of about 1 to about 5 μg/ml can be targeted by the present method. Steady-state values of Cₘₐₓ and Cₘᵢₙ can be established in a human PK study, for example conducted according to standard protocols including but not limited to those acceptable to a regulatory agency such as the U.S. Food and Drug Administration (FDA).

Administration according to the present embodiment can be with or without food, i.e., in a non-fasting or fasting condition. However, as compositions of the invention can show a positive food effect, it is generally preferred to administer the present compositions to a non-fasting patient.

Compositions of the invention are suitable for use in monotherapy or in combination therapy, for example with other chemotherapeutics or with ionizing radiation. A particular advantage of the present invention is that it permits once-daily oral administration, a regimen which is convenient for the patient who is undergoing treatment with other orally administered drugs on a once-daily regimen. Oral administration is easily accomplished by the patient him/herself or by a caregiver in the patient's home; it is also a convenient route of administration for patients in a hospital or residential care setting.

Combination therapies illustratively include administration of a composition of the present invention, for example such a composition comprising ABT-263, concomitantly with one or more of bortezomib, carboplatin, cisplatin, cyclophosphamide, dacarbazine, dexamethasone, docetaxel, doxorubicin, etoposide, fludarabine, irinotecan, paclitaxel, rapamycin, rituximab, vincristine and the like, for example with a polytherapy such as CHOP (cyclophosphamide+doxorubicin+vincristine+prednisone), RCVP (rituximab+cyclophosphamide+vincristine+prednisone), R-CHOP (rituximab+CHOP) or DA-EPOCH-R (dose-adjusted etoposide, prednisone, vincristine, cyclophosphamide, doxorubicin and rituximab).

A composition of the invention, for example such a composition comprising ABT-263, can be administered in combination therapy with one or more therapeutic agents that include, but are not limited to, alkylating agents, angiogenesis inhibitors, antibodies, antimetabolites, antimitotics, antiproliferatives, antivirals, aurora kinase inhibitors, other apoptosis promoters (for example, Bc1-xL, Bc1-w and Bf1-1 inhibitors), activators of a death receptor pathway, Bcr-Abl kinase inhibitors, BiTE (bi-specific T-cell engager) antibodies, antibody-drug conjugates, biological response modifiers, cyclin-dependent kinase (CDK) inhibitors, cell cycle inhibitors, cyclooxygenase-2 (COX-2) inhibitors, dual variable domain binding proteins (DVDs), human epidermal growth factor receptor 2 (ErbB2 or HER/2neu) receptor inhibitors, growth factor inhibitors, heat shock protein (HSP)-90 inhibitors, histone deacetylase (HDAC) inhibitors, hormonal therapies, immunologicals, inhibitors of apoptosis proteins (IAPs), intercalating antibiotics, kinase inhibitors, kinesin inhibitors, JAK2 inhibitors, mammalian target of rapamycin (mTOR) inhibitors, microRNAs, mitogen-activated extracellular signal-regulated kinase (MEK) inhibitors, multivalent binding proteins, non-steroidal anti-inflammatory drugs (NSAIDs), poly-ADP (adenosine diphosphate)-ribose polymerase (PARP) inhibitors, platinum chemotherapeutics, polo-like kinase (PLK) inhibitors, phosphoinositide-3 kinase (PI3K) inhibitors, proteasome inhibitors, purine analogs, pyrimidine analogs, receptor tyrosine kinase inhibitors, retinoids, deltoids, plant alkaloids, small inhibitory ribonucleic acids (siRNAs), topoisomerase inhibitors, ubiquitin ligase inhibitors, and the like.

BiTE antibodies are bi-specific antibodies that direct T-cells to attack cancer cells by simultaneously binding the two cells. The T-cell then attacks the target cancer cell. Examples of BiTE antibodies include, but are not limited to, adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B. In this regard, Bc1-2 has been shown to attenuate the induction of apoptosis by both perforin and granzyme B. These data suggest that inhibition of Bc1-2 could enhance the cytotoxic effects elicited by T-cells when targeted to cancer cells (Sutton et al. (1997) J. Immunol. 158:5783-5790).

SiRNAs are molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand. For example, siRNAs targeting Mc1-1 have been shown to enhance the activity of ABT-263 or ABT-737 in various tumor cell lines (Tse et al. (2008) Cancer Res. 68:3421-3428 and references therein).

Multivalent binding proteins are binding proteins comprising two or more antigen binding sites. Multivalent binding proteins are engineered to have the three or more antigen binding sites and are generally not naturally occurring antibodies. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific (i.e., capable of binding one antigen) or multispecific (i.e., capable of binding two or more antigens). DVD binding proteins comprising two heavy-chain DVD polypeptides and two light-chain DVD polypeptides are referred to as DVD Ig's. Each half of a DVD Ig comprises a heavy-chain DVD polypeptide, a light-chain DVD polypeptide, and two antigen binding sites. Each binding site comprises a heavy-chain variable domain and a light-chain variable domain with a total of 6 CDRs involved in antigen binding per antigen binding site.

Alkylating agents include altretamine, AMD-473, AP-5280, apaziquone, bendamustine, brostallicin, busulfan, carboquone, carmustine (BCNU), chlorambucil, Cloretazine™ (laromustine, VNP 40101M), cyclophosphamide, dacarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, treosulfan, trofosfamide and the like.

Angiogenesis inhibitors include epidermal growth factor receptor (EGFR) inhibitors, endothelial-specific receptor tyrosine kinase (Tie-2) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs, vascular endothelial growth factor receptor tyrosine kinase (VEGFR) inhibitors and the like.

Antimetabolites include Alimta™ (pemetrexed disodium, LY231514, MTA), 5-azacitidine, Xeloda™ (capecitabine), carmofur, Leustat™ (cladribine), clofarabine, cytarabine, cytarabine ocfosfate, cytosine arabinoside, decitabine, deferoxamine, doxifluridine, eflornithine, EICAR (5-ethynyl-1-β-D-ribofuranosylimidazole-4-carboxamide), enocitabine, ethenylcytidine, fludarabine, 5-fluorouracil (5-FU) alone or in combination with leucovorin, Gemzar™ (gemcitabine), hydroxyurea, Alkeran™ (melphalan), mercaptopurine, 6-mercaptopurine riboside, methotrexate, mycophenolic acid, nelarabine, nolatrexed, ocfosfate, pelitrexol, pentostatin, raltitrexed, ribavirin, S-1, triapine, trimetrexate, TS-1, tiazofurin, tegafur, vidarabine, UFT and the like.

Antivirals include ritonavir, hydroxychloroquine and the like.

Aurora kinase inhibitors include ABT-348, AZD-1152, MLN-8054, VX-680, aurora A-specific kinase inhibitors, aurora B-specific kinase inhibitors, pan-aurora kinase inhibitors and the like.

Bc1-2 family protein inhibitors other than ABT-263 or compounds of Formula I herein include AT-101 ((−)gossypol), Genasense™ Bc1-2-targeting antisense oligonucleotide (G3139 or oblimersen), IPI-194, IPI-565, ABT-737, GX-070 (obatoclax) and the like.

Bcr-Abl kinase inhibitors include dasatinib (BMS-354825), Gleevec™ (imatinib) and the like.

CDK inhibitors include AZD-5438, BMI-1040, BMS-387032, CVT-2584, flavopyridol, GPC-286199, MCS-5A, PD0332991, PHA-690509, seliciclib (CYC-202 or R-roscovitine), ZK-304709 and the like.

COX-2 inhibitors include ABT-963, Arcoxia™ (etoricoxib), Bextra™ (valdecoxib), BMS-347070, Celebrex™ (celecoxib), COX-189 (lumiracoxib), CT-3, Deramaxx™ (deracoxib), JTE-522,4-methyl-2-(3,4-dimethylphenyl)-1-(4-sulfamoylphenyl)-1H-pyrrole, MK-663 (etoricoxib), NS-398, parecoxib, RS-57067, SC-58125, SD-8381, SVT-2016, S-2474, T-614, Vioxx™ (rofecoxib) and the like.

EGFR inhibitors include ABX-EGF, anti-EGFR immunoliposomes, EGF-vaccine, EMD-7200, Erbitux™ (cetuximab), HR3, IgA antibodies, Iressa™ (gefitinib), Tarceva™ (erlotinib or OSI-774), TP-38, EGFR fusion protein, Tykerb™ (lapatinib) and the like.

ErbB2 receptor inhibitors include CP-724714, CI-1033 (canertinib), Herceptin™ (trastuzumab), Tykerb™ (lapatinib), Omnitarg™ (2C4, petuzumab), TAK-165, GW-572016 (ionafamib), GW-282974, EKB-569, PI-166, dHER2 (HER2 vaccine), APC-8024 (HER2 vaccine), anti-HER/2neu bispecific antibody, B7.her2IgG3, AS HER2 trifunctional bispecific antibodies, mAB AR-209, mAB 2B-1 and the like.

Histone deacetylase inhibitors include depsipeptide, LAQ-824, MS-275, trapoxin, suberoylanilide hydroxamic acid (SAHA), TSA, valproic acid and the like.

HSP-90 inhibitors include 17AAG, CNF-101, CNF-1010, CNF-2024, 17-DMAG, geldanamycin, IPI-504, KOS-953, Mycograb™ (human recombinant antibody to HSP-90), nab-17AAG, NCS-683664, PU24FC1, PU-3, radicicol, SNX-2112, STA-9090, VER-49009 and the like.

Inhibitors of apoptosis proteins include HGS-1029, GDC-0145, GDC-0152, LCL-161, LBW-242 and the like.

Antibody-drug conjugates include anti-CD22-MC-MMAF, anti-CD22-MC-MMAE, anti-CD22-MCC-DM1, CR-011-vcMMAE, PSMA-ADC, MEDI-547, SGN-19A, SGN-35, SGN-75 and the like.

Activators of death receptor pathway include TRAIL and antibodies or other agents that target TRAIL or death receptors (e.g., DR4 and DR5) such as apomab, conatumumab, ETR2-ST01, GDC0145 (lexatumumab), HGS-1029, LBY-135, PRO-1762, trastuzumab and the like.

Kinesin inhibitors include Eg5 inhibitors such as AZD-4877 and ARRY-520, CENPE inhibitors such as GSK-923295A, and the like.

JAK2 inhibitors include CEP-701 (lesaurtinib), XL019, NCB-018424 and the like.

MEK inhibitors include ARRY-142886, ARRY-438162, PD-325901, PD-98059 and the like.

mTOR inhibitors include AP-23573, CCI-779, everolimus, RAD-001, rapamycin, temsirolimus, ATP-competitive TORC1/TORC2 inhibitors, including PI-103, PP242, PP30 and Torin 1, and the like.

Non-steroidal anti-inflammatory drugs include Amigesic™ (salsalate), Dolobid™ (diflunisal), Motrin™ (ibuprofen), Orudis™ (ketoprofen), Relafen™ (nabumetone), Feldene™ (piroxicam), ibuprofen cream, Aleve™ and Naprosyn™ (naproxen), Voltaren™ (diclofenac), Indocin™ (indomethacin), Clinoril™ (sulindac), Tolectin™ (tolmetin), Lodine™ (etodolac), Toradol™ (ketorolac), Daypro™ (oxaprozin) and the like.

PDGFR inhibitors include CP-673451, CP-868596 and the like.

Platinum chemotherapeutics include cisplatin, Eloxatin™ (oxaliplatin), eptaplatin, lobaplatin, nedaplatin, Paraplatin™ (carboplatin), picoplatin, satraplatin and the like.

Polo-like kinase inhibitors include BI-2536 and the like.

Phosphoinositide-3 kinase inhibitors include wortmannin, LY-294002, XL-147, CAL-120, ONC-21, AEZS-127, ETP-45658, PX-866, GDC-0941, BGT226, BEZ235, XL765 and the like.

Thrombospondin analogs include ABT-510, ABT-567, ABT-898, TSP-1 and the like.

VEGFR inhibitors include Avastin™ (bevacizumab), ABT-869, AEE-788, Angiozyme™ (a ribozyme that inhibits angiogenesis (Ribozyme Pharmaceuticals (Boulder, Colo.) and Chiron (Emeryville, Calif.)), axitinib (AG-13736), AZD-2171, CP-547632, IM-862, Macugen™ (pegaptanib), Nexavar™ (sorafenib, BAY43-9006), pazopanib (GW-786034), vatalanib (PTK-787 or ZK-222584), Sutent™ (sunitinib or SU-11248), VEGF trap, Zactima™ (vandetanib or ZD-6474) and the like.

Antibiotics include intercalating antibiotics such as aclarubicin, actinomycin D, amrubicin, annamycin, Adriamycin™ (doxorubicin), Blenoxane™ (bleomycin), daunorubicin, Caelyx™ and Myocet™ (liposomal doxorubicin), elsamitrucin, epirubicin, glarubicin, idarubicin, mitomycin C, nemorubicin, neocarzinostatin, peplomycin, pirarubicin, rebeccamycin, stimalamer, streptozocin, Valstar™ (valrubicin), zinostatin and the like.

Topoisomerase inhibitors include aclarubicin, 9-aminocamptothecin, amonafide, amsacrine, becatecarin, belotecan, BN-80915, Camptosar™ (irinotecan hydrochloride), camptothecin, Cardioxane™ (dexrazoxane), diflomotecan, edotecarin, Ellence™ and Pharmorubicin™ (epirubicin), etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

Antibodies include Avastin™ (bevacizumab), CD40-specific antibodies, chTNT-1/B, denosumab, Erbitux™ (cetuximab), Humax-CD4™ (zanolimumab), IGF1R-specific antibodies, lintuzumab, Panorex™ (edrecolomab), Rencarex™ (WX G250), Rituxan™ (rituximab), ticilimumab, trastuzumab, CD20 antibodies types I and II and the like.

Hormonal therapies include Arimidex™ (anastrozole), Aromasin™ (exemestane), arzoxifene, Casodex™ (bicalutamide), Cetrotide™ (cetrorelix), degarelix, deslorelin, Desopan™ (trilostane), dexamethasone, Drogenil™ (flutamide), Evista™ (raloxifene), Afema™ (fadrozole), Fareston™ (toremifene), Faslodex™ (fulvestrant), Femara™ (letrozole), formestane, glucocorticoids, Hectorol™ (doxercalciferol), Renagel™ (sevelamer carbonate), lasofoxifene, leuprolide acetate, Megace™ (megestrol), Mifeprex™ (mifepristone), Nilandron™ (nilutamide), tamoxifen including Nolvadex™ (tamoxifen citrate), Plenaxis™ (abarelix), prednisone, Propecia™ (finasteride), rilostane, Suprefact™ (buserelin), luteinizing hormone releasing hormone (LHRH) including Trelstar™ (triptorelin), histrelin including Vantas™ (histrelin implant), Modrastane™ (trilostane), Zoladex™ (goserelin) and the like.

Deltoids and retinoids include seocalcitol (EB1089 or CB1093), lexacalcitol (KH1060), fenretinide, Panretin™ (alitretinoin), tretinoin including Atragen™ (liposomal tretinoin), Targretin™ (bexarotene), LGD-1550 and the like.

PARP inhibitors include ABT-888, olaparib, KU-59436, AZD-2281, AG-014699, BSI-201, BGP-15, INO-1001, ONO-2231 and the like.

Plant alkaloids include vincristine, vinblastine, vindesine, vinorelbine and the like.

Proteasome inhibitors include Velcade™ (bortezomib), MG132, NPI-0052, PR-171 and the like.

Examples of immunologicals include interferons and other immune-enhancing agents. Interferons include interferon alpha, interferon alpha-2a, interferon alpha-2b, interferon beta, interferon gamma-1a, Actimmune™ (interferon gamma-1b), interferon gamma-n1, combinations thereof and the like. Other agents include Alfaferone (IFN-α), BAM-002 (oxidized glutathione), Beromun™ (tasonermin), Bexxar™ (tositumomab), Campath™ (alemtuzumab), CTLA4 (cytotoxic lymphocyte antigen 4), dacarbazine, denileukin, epratuzumab, Granocyte™ (lenograstim), lentinan, leukocyte alpha interferon, imiquimod, MDX-010 (anti-CTLA-4), melanoma vaccine, mitumomab, molgramostim, Mylotarg™ (gemtuzumab ozogamicin), Neupogen™ (filgrastim), OncoVAC-CL, Ovarex™ (oregovomab), pemtumomab (Y-muHMFG1), Provenge™ (sipuleucel-T), sargaramostim, sizofuran, teceleukin, Theracys™ (BCG or Bacillus Calmette-Guerin), ubenimex, Virulizin™ (immunotherapeutic, Lorus Pharmaceuticals), Z-100 (Specific Substance of Maruyama or SSM), WF-10 (tetrachlorodecaoxide or TCDO), Proleukin™ (aldesleukin), Zadaxin™ (thymalfasin), Zenapax™ (daclizumab), Zevalin™ (90Y-ibritumomab tiuxetan) and the like.

Biological response modifiers are agents that modify defense mechanisms of living organisms or biological responses, such as survival, growth or differentiation of tissue cells to direct them to have anti-tumor activity, and include krestin, lentinan, sizofuran, picibanil, PF-3512676 (CpG-8954), ubenimex and the like.

Pyrimidine analogs include cytarabine (cytosine arabinoside, ara C or arabinoside C), doxifluridine, Fludara™ (fludarabine), 5-FU (5-fluorouracil), floxuridine, Gemzar™ (gemcitabine), Tomudex™ (raltitrexed), triacetyluridine, Troxatyl™ (troxacitabine) and the like.

Purine analogs include Lanvis™ (thioguanine), Purinethol™ (mercaptopurine) and the like.

Antimitotic agents include batabulin, epothilone D (KOS-862), N-(2-((4-hydroxy-phenyl)amino)pyridin-3-yl)-4-methoxybenzenesulfonamide, ixabepilone (BMS-247550), paclitaxel, Taxotere™ (docetaxel), larotaxel (PNU-100940, RPR-109881 or XRP-9881), patupilone, vinflunine, ZK-EPO (synthetic epothilone) and the like.

Ubiquitin ligase inhibitors include MDM2 inhibitors such as nutlins, NEDD8 inhibitors such as MLN4924, and the like.

Compositions of this invention can also be used as radiosensitizers that enhance the efficacy of radiotherapy. Examples of radiotherapy include, but are not limited to, external beam radiotherapy (XBRT), teletherapy, brachytherapy, sealed-source radiotherapy, unsealed-source radiotherapy and the like.

Additionally or alternatively, a composition of the present invention can be administered in combination therapy with one or more antitumor or chemotherapeutic agents selected from Abraxane™ (ABI-007), ABT-100 (farnesyl transferase inhibitor), Advexin™ (Ad5CMV-p53 vaccine or contusugene ladenovec), Altocor™ or Mevacor™ (lovastatin), Ampligen™ (poly(I)-poly(C12U), a synthetic RNA), Aptosyn™ (exisulind), Aredia™ (pamidronic acid), arglabin, L-asparaginase, atamestane (1-methyl-3,17-dione-androsta-1,4-diene), Avage™ (tazarotene), AVE-8062 (combretastatin derivative), BEC2 (mitumomab), cachectin or cachexin (tumor necrosis factor), Canvaxin™ (melanoma vaccine), CeaVac™ (cancer vaccine), Celeuk™ (celmoleukin), histamine including Ceplene™ (histamine dihydrochloride), Cervarix™ (AS04 adjuvant-adsorbed human papilloma virus (HPV) vaccine), CHOP (Cytoxan™ (cyclophosphamide)+Adriamycin™ (doxorubicin)+Oncovin™ (vincristine)+prednisone), combretastatin A4P, Cypat™ (cyproterone), DAB(389)EGF (catalytic and translocation domains of diphtheria toxin fused via a His-Ala linker to human epidermal growth factor), dacarbazine, dactinomycin, Dimericine™ (T4N5 liposome lotion), 5,6-dimethylxanthenone-4-acetic acid (DMXAA), discodermolide, DX-8951f (exatecan mesylate), eniluracil (ethynyluracil), squalamine including Evizon™ (squalamine lactate), enzastaurin, EPO-906 (epothilone B), Gardasil™ (quadrivalent human papilloma virus (Types 6, 11, 16, 18) recombinant vaccine), Gastrimmune™, Genasense™ (oblimersen), GMK (ganglioside conjugate vaccine), GVAX™ (prostate cancer vaccine), halofuginone, histerelin, hydroxycarbamide, ibandronic acid, IGN-101, IL-13-PE38, IL-13-PE38QQR (cintredekin besudotox), IL-13-pseudomonas exotoxin, interferon-α, interferon-γ, Junovan™ and Mepact™ (mifamurtide), lonafarnib, 5,10-methylenetetrahydrofolate, miltefosine (hexadecyl-phosphocholine), Neovastat™ (AE-941), Neutrexin™ (trimetrexate glucuronate), Nipent™ (pentostatin), Onconase™ (ranpirnase, a ribonuclease enzyme), Oncophage™ (vitespen, melanoma vaccine treatment), OncoVAX™ (IL-2 vaccine), Orathecin™ (rubitecan), Osidem™ (antibody-based cell drug), Ovarex™ MAb (murine monoclonal antibody), paclitaxel albumin-stabilized nanoparticle, paclitaxel, Pandimex™ (aglycone saponins from ginseng comprising 20(S)-protopanaxadiol (aPPD) and 20(S)-protopanaxatriol (aPPT)), panitumumab, Panvac™-VF (investigational cancer vaccine), pegaspargase, peginterferon alfa (PEG interferon A), phenoxodiol, procarbazine, rebimastat, Removab™ (catumaxomab), Revlimid™ (lenalidomide), RSR13 (efaproxiral), Somatuline™ LA (lanreotide), Soriatane™ (acitretin), staurosporine (Streptomyces staurospores), talabostat (PT100), Targretin™ (bexarotene), Taxoprexin™ (docosahexaenoic acid (DHA)+paclitaxel), Telcyta™ (canfosfamide, TLK-286), Temodar™ (temozolomide), tesmilifene, tetrandrine, thalidomide, Theratope™ (STn-KLH vaccine), Thymitaq™ (nolatrexed dihydrochloride), TNFerade™ (adenovector: DNA carrier containing the gene for tumor necrosis factor-α), Tracleer™ or Zavesca™ (bosentan), TransMID-107R™ (KSB-311, diphtheria toxins), tretinoin (retin-A), Trisenox™ (arsenic trioxide), Ukrain™ (derivative of alkaloids from the greater celandine plant), Virulizin™, Vitaxin™ (anti-αvβ3 antibody), Xcytrin™ (motexafin gadolinium), Xinlay™ (atrasentan), Xyotax™ (paclitaxel poliglumex), Yondelis™ (trabectedin), ZD-6126 (N-acetylcolchinol-O-phosphate), Zinecard™ (dexrazoxane), zoledronic acid, zorubicin and the like.

In one embodiment, a composition of the invention, for example such a composition comprising ABT-263, is administered in a therapeutically effective amount to a subject in need thereof to treat a disease during which is overexpressed one or more of antiapoptotic Bc1-2 protein, antiapoptotic Bc1-X_{L} protein and antiapoptotic Bc1-w protein.

In another embodiment, a composition of the invention, for example such a composition comprising ABT-263, is administered in a therapeutically effective amount to a subject in need thereof to treat a disease of abnormal cell growth and/or dysregulated apoptosis.

Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In a more particular embodiment, a composition of the invention, for example such a composition comprising ABT-263, is administered in a therapeutically effective amount to a subject in need thereof to treat bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer or spleen cancer.

According to any of these embodiments, the composition can be administered in monotherapy or in combination therapy with one or more additional therapeutic agents.

For example, a method for treating mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof in a subject comprises administering to the subject therapeutically effective amounts of (a) a composition of the invention, for example such a composition comprising ABT-263, and (b) one or more of etoposide, vincristine, CHOP, rituximab, rapamycin, R-CHOP, RCVP, DA-EPOCH-R or bortezomib.

In particular embodiments, a composition of the invention, for example such a composition comprising ABT-263, is administered in a therapeutically effective amount to a subject in need thereof in combination therapy with etoposide, vincristine, CHOP, rituximab, rapamycin, R-CHOP, RCVP, DA-EPOCH-R or bortezomib in a therapeutically effective amount, for treatment of a lymphoid malignancy such as B-cell lymphoma or non-Hodgkin's lymphoma.

The present invention also provides a method for maintaining in bloodstream of a human cancer patient a therapeutically effective plasma concentration of ABT-263 and/or one or more metabolites thereof, comprising administering to the subject a nanoparticulate suspension that comprises ABT-263 or a pharmaceutically acceptable salt, prodrug, salt of a prodrug or metabolite thereof, more particularly a crystalline salt of ABT-263, for example ABT-263 bis-HCl, in a dosage amount of about 50 to about 500 mg ABT-263 free base equivalent per day, at an average dosage interval of about 3 hours to about 7 days.

What constitutes a therapeutically effective plasma concentration depends inter alia on the particular cancer present in the patient, the stage, severity and aggressiveness of the cancer, and the outcome sought (e.g., stabilization, reduction in tumor growth, tumor shrinkage, reduced risk of metastasis, etc.). It is strongly preferred that, while the plasma concentration is sufficient to provide benefit in terms of treating the cancer, it should not be sufficient to provoke an adverse side-effect to an unacceptable or intolerable degree.

For treatment of cancer in general and of a lymphoid malignancy such as non-Hodgkin's lymphoma in particular, the plasma concentration of ABT-263 should in most cases be maintained in a range of about 0.5 to about 10 μg/ml. Thus, during a course of ABT-263 therapy, the steady-state Cₘₐₓ should in general not exceed about 10 μg/ml, and the steady-state Cₘᵢₙ should in general not fall below about 0.5 μg/ml. It will further be found desirable to select, within the ranges provided above, a daily dosage amount and average dosage interval effective to provide a Cₘₐₓ/Cₘᵢₙ ratio not greater than about 5, for example not greater than about 3, at steady-state. It will be understood that longer dosage intervals will tend to result in greater Cₘₐₓ/Cₘᵢₙ ratios. Illustratively, at steady-state, an ABT-263 Cₘₐₓ of about 3 to about 8 μg/ml and Cₘᵢₙ of about 1 to about 5 μg/ml can be targeted by the present method.

A daily dosage amount effective to maintain a therapeutically effective ABT-263 plasma level is, according to the present embodiment, about 50 to about 500 mg. In most cases a suitable daily dosage amount is about 200 to about 400 mg. Illustratively, the daily dosage amount can be for example about 50, about 100, about 150, about 200, about 250, about 300, about 350, about 400, about 450 or about 500 mg.

An average dosage interval effective to maintain a therapeutically effective ABT-263 plasma level is, according to the present embodiment, about 3 hours to about 7 days. In most cases a suitable average dosage interval is about 8 hours to about 3 days, or about 12 hours to about 2 days. A once-daily (q.d.) administration regimen is often suitable.

For the present embodiment, ABT-263 is illustratively present in the pharmaceutical composition in the form of ABT-263 bis-HCl or other crystalline ABT-263 salt. Any ABT-263 composition of the present invention, as defined more fully above, can be used.

As in other embodiments, administration according to the present embodiment can be with or without food, i.e., in a non-fasting or fasting condition. It is generally preferred to administer the present compositions to a non-fasting patient.

### EXAMPLES

The following examples are merely illustrative, and do not limit this disclosure in any way.

All ABT-263 amounts, including concentrations and doses, given in the examples are expressed as free base equivalent doses unless expressly stated otherwise. Where ABT-263 is used as bis-HCl salt, 1.076 mg ABT-263 bis-HCl provides 1 mg ABT-263 free base equivalent.

### Example 1

### Preparation of an Illustrative Nanoparticulate Suspension

ABT-263 nanoparticulate suspension formulations were prepared by high-pressure homogenization as described below. The formulations had the following compositions (all percentages expressed as weight/volume) in water:

Formulation I (Comparative)

| ABT-263 bis-HCl | 5% (4.65% free base equivalent) |
|---|---|
| poloxamer 188 | 3% |

Formulation II (Illustrative of the Invention)

| ABT-263 bis-HCl | 5% (4.65% free base equivalent) |
|---|---|
| poloxamer 188 | 3% |
| NaHCO₃ | 8.4% |

Aqueous solutions were prepared containing the indicated amount of poloxamer 188 (Pluronic™ F68) and, in the case of Formulation II, sodium bicarbonate (NaHCO₃). Crystalline ABT-263 bis-HCl in an amount sufficient to provide a 5% weight/volume (50 mg/ml) suspension was dispersed in each aqueous solution using a Sonifier™ homogenizer (Branson Ultrasonic, Danbury, Conn.). The resulting dispersion was then added to the sample reservoir of a Microfluidizer™ M-110L processor (Microfluidics International Corp., Newton, Mass.) and processed at 12,000 psi (approximately 82.5 MPa) for 2 hours. The sample temperature was maintained throughout at a temperature of 20±2° C. by running the dispersion through a heat exchanger immersed in a water bath connected to a chiller.

The suspensions so obtained (Formulations I and II) were subjected to particle size measurement immediately upon preparation and after storage for 14 days at 5° C. (see Example 2). Formulation II was submitted to an oral pharmacokinetic (PK) study in dogs (see Example 3).

### Example 2

### Effect of Sodium Bicarbonate on Particle Size Stability of Nanosuspensions

Formulations I and II were compared as to their particle size distribution (D₉₀ and D₅₀). Particle size measurement was performed immediately upon preparation of the suspensions (t=0) and after storage for 14 days at 5° C. In addition particle size was measured at t=0 for suspensions following dilution of 1 ml of each suspension in 20 ml 0.9% sodium chloride (NaCl) solution. Data are given in Table 1.

TABLE 1
D₉₀ and D₅₀ particle sizes (μm)
of nanosuspension Formulations I and II

| | |
|---|---|
| Formulation I | Formulation II |
| (no NaHCO₃) | (8.4% NaHCO₃) |

| | D₉₀ | D₅₀ | D₉₀ | D₅₀ |
|---|---|---|---|---|
| t = 0 | 1.126 | 0.490 | 0.605 | 0.291 |
| 14 d at 5° C. | 1.214 | 0.570 | 0.621 | 0.295 |
| t = 0 in 0.9% NaCl | 1.712 | 0.886 | 0.596 | 0.295 |

### Example 3

### Pharmacokinetics of an Illustrative Nanosuspension

Single-dose pharmacokinetics of Formulation II of Example 1 were evaluated in non-fasted beagle dogs (n=4) after a 5 mg/kg oral dose. The formulation was administered in two ways: by oral gavage and in a capsule. Formulation II was also administered to histamine-pretreated fasted dogs (n=4), by oral gavage only. For comparative purposes, a solution formulation of ABT-263 bis-HCl in a lipid medium (Formulation C, prepared from ABT-263 bis-HCl powder dissolved to a concentration of 25 mg/ml in a 90:10 mixture of Phosal 53 MCT™ and ethanol) was administered to non-fasted dogs. Formulation C has been used to evaluate ABT-263 in clinical studies. Phosal 53 MCT™ is a proprietary blend supplied by Phospholipid GmbH and contains 53% phosphatidylcholine and 29% medium chain triglycerides.

Serial heparinized blood samples were obtained from a jugular vein of each animal prior to dosing and 0.25, 0.5, 1, 1.5, 2, 3, 4, 6, 9, 12, 15 and 24 hours after administration. Plasma was separated by centrifugation (2,000 rpm for 10 minutes at approximately 4° C.) and ABT-263 was isolated using protein precipitation with acetonitrile.

ABT-263 and an internal standard were separated from each other and from co-extracted contaminants on a 50×3 mm Keystone Betasil CN™ 5 μm column with an acetonitrile/0.1% trifluoroacetic acid mobile phase (50:50 by volume) at a flow rate of 0.7 ml/min. Analysis was performed on a Sciex API3000™ biomolecular mass analyzer with a heated nebulizer interface. ABT-263 and internal standard peak areas were determined using Sciex MacQuan™ software. The plasma drug concentration of each sample was calculated by least squares linear regression analysis (non-weighted) of the peak area ratio (parent/internal standard) of the spiked plasma standards versus concentration. The plasma concentration data were submitted to multi-exponential curve fitting using WinNonlin 3 (Pharsight).

The area under the plasma concentration-time curve from 0 to t hours (time of the last measured plasma concentration, which here is 24 hours) after dosing (AUC₀₋₂₄) was calculated using the linear trapezoidal rule for the plasma concentration-time profiles.

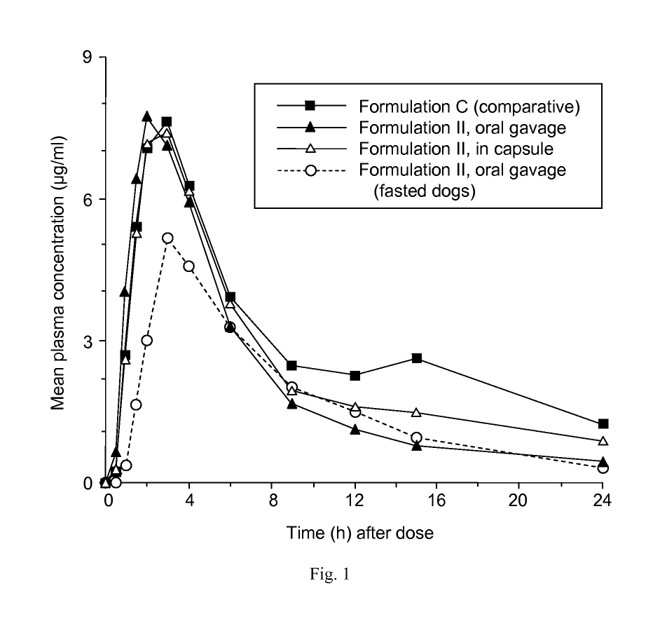

Mean plasma concentrations over 24 hours after dosing are shown in FIG. 1.

Calculated mean PK parameters are summarized in Table 2.

TABLE 2
PK parameters (mean ± SEM) in dogs
(non-fasted unless otherwise indicated)

| | Cₘₐₓ | Tₘₐₓ | AUC₀₋₂₄ | Bioavailability |
|---|---|---|---|---|
| | (μg/ml) | (h) | (μg · h/ml) | F (%) |
| Formulation C | 9.09 ± 1.33 | 6.3 ± 1.6 | 54.5 ± 6.3 | 22.4 ± 2.6 |
| (comparative) | | | | |
| Formulation II, | 7.78 ± 0.35 | 2.3 ± 0.3 | 45.2 ± 2.6 | 19.9 ± 1.2 |
| oral gavage | | | | |
| Formulation II, | 7.52 ± 2.46 | 3.0 ± 0.4 | 48.3 ± 12.4 | 21.3 ± 5.5 |
| in capsule | | | | |
| Formulation II, | 5.56 ± 0.46 | 3.3 ± 0.3 | 35.6 ± 0.6 | 15.7 ± 0.2 |
| oral gavage | | | | |
| (fasted dogs) | | | | |

What is claimed is:

1. A liquid pharmaceutical composition comprising an aqueous medium having suspended therein a solid particulate compound having a D₉₀ particle size not greater than about 3 μm; wherein the compound is of Formula I: 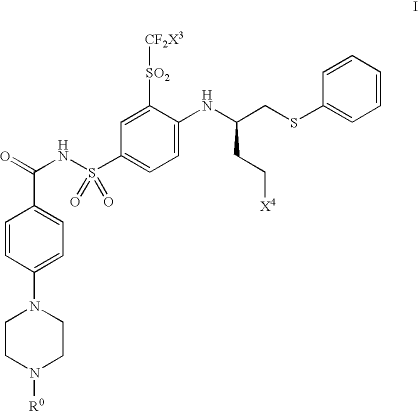
where:
X³ is chloro or fluoro; and
(1) X⁴ is azepan-1-yl, morpholin-4-yl, 1,4-oxazepan-4-yl, pyrrolidin-1-yl, —N(CH₃)₂, —N(CH₃)(CH(CH₃)₂), 7-azabicyclo[2.2.1]heptan-7-yl or 2-oxa-5-azabicyclo[2.2.1]hept-5-yl; and R⁰ is 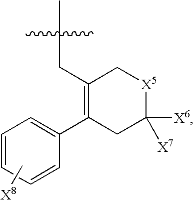
where
X⁵ is —CH₂—, —C(CH₃)₂— or —CH₂CH₂—;
X⁶ and X⁷ are both —H or both methyl; and
X⁸ is fluoro, chloro, bromo or iodo;
or
(2) X⁴ is azepan-1-yl, morpholin-4-yl, pyrrolidin-1-yl, —N(CH₃)(CH(CH₃)₂) or 7-azabicyclo[2.2.1]heptan-7-yl; and R⁰ is 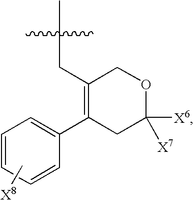
where X⁶, X⁷ and X⁸ are as above; or
(3) X⁴ is morpholin-4-yl or —N(CH₃)₂; and R⁰ is 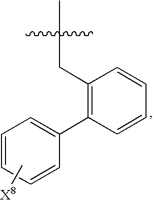
where X⁸ is as above;
or a pharmaceutically acceptable salt, prodrug, salt of a prodrug or metabolite thereof; and wherein the aqueous medium further comprises at least one pharmaceutically acceptable surfactant and at least one pharmaceutically acceptable basifying agent in amounts that are effective together to inhibit particle size increase.

2. The composition of claim 1, wherein the compound has a D₉₀ particle size not greater than about 800 nm and/or a D₅₀ particle size not greater than about 350 nm.

3. The composition of claim 1, wherein the drug compound is present in an amount of about 20 to about 200 mg/ml.

4. The composition of claim 1, wherein the at least one surfactant is selected from the group consisting of benzalkonium chloride, benzethonium chloride, cetylpyridinium chloride, dioctyl sodium sulfosuccinate, polyoxyethylene alkylphenyl ethers, nonoxynol 9, nonoxynol 10, octoxynol 9, poloxamers, poloxamer 188, poloxamer 237, polyoxyethylene fatty acid glycerides, polyoxyethylene fatty acid oils, polyoxyethylene (8) caprylic/capric mono- and diglycerides, polyoxyethylene (35) castor oil, polyoxyethylene (40) hydrogenated castor oil, polyoxyethylene alkyl ethers, ceteth-10, laureth-4, laureth-23, oleth-2, oleth-10, oleth-20, steareth-2, steareth-10, steareth-20, steareth-100, polyoxyethylene (20) cetostearyl ether, polyoxyethylene fatty acid esters, polyoxyethylene (20) stearate, polyoxyethylene (40) stearate, polyoxyethylene (100) stearate, sorbitan esters, sorbitan monolaurate, sorbitan monooleate, sorbitan monopalmitate, sorbitan monostearate, polyoxyethylene sorbitan esters, polysorbate 20, polysorbate 80, propylene glycol fatty acid esters, propylene glycol laurate, sodium lauryl sulfate, oleic acid, sodium oleate, triethanolamine oleate, glyceryl fatty acid esters, glyceryl monooleate, glyceryl monostearate, glyceryl palmitostearate, TPGS, tyloxapol and combinations thereof.

5. The composition of claim 1, wherein the at least one surfactant is present in a total surfactant amount of about 10 to about 100 mg/ml.

6. The composition of claim 1, wherein the at least one basifying agent comprises sodium bicarbonate.

7. The composition of claim 6, wherein the sodium bicarbonate is present in an amount of about 20 to about 200 mg/ml.

8. The composition of claim 1, wherein the compound is ABT-263 or a crystalline salt thereof.

9. The composition of claim 1, wherein the compound is ABT-263 free base, ABT-263 bis-HCl salt or a combination thereof.

10. The composition of claim 9, wherein the at least one surfactant comprises a poloxamer and is present in a total surfactant amount of about 10 to about 100 mg/ml.

11. The composition of claim 9, wherein the at least one surfactant comprises poloxamer 188 and is present in a total surfactant amount of about 15 to about 60 mg/ml.

12. The composition of claim 9, wherein the at least one basifying agent comprises sodium bicarbonate and is present in an amount of about 20 to about 200 mg/ml.

13. The composition of claim 9, wherein the at least one basifying agent comprises sodium bicarbonate and is present in an amount of about 40 to about 160 mg/ml.

14. The composition of claim 1, wherein the aqueous medium is a saline medium.

15. The composition of claim 1 that is adapted for parenteral or oral administration.

16. A solid pharmaceutical composition comprising a compound of Formula I 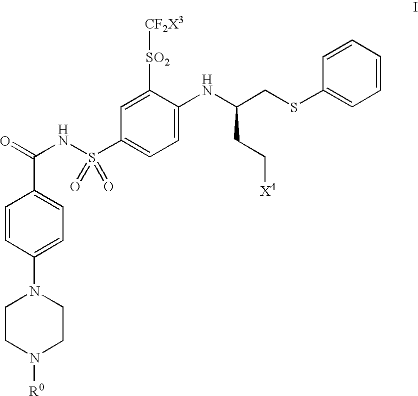
where:
X³ is chloro or fluoro; and
(1) X⁴ is azepan-1-yl, morpholin-4-yl, 1,4-oxazepan-4-yl, pyrrolidin-1-yl, —N(CH₃)₂, —N(CH₃)(CH(CH₃)₂), 7-azabicyclo[2.2.1]heptan-7-yl or 2-oxa-5-azabicyclo[2.2.1]hept-5-yl; and R⁰ is 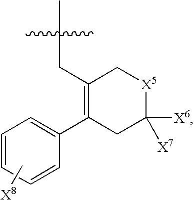
where
X⁵ is —CH₂—, —C(CH₃)₂— or —CH₂CH₂—;
X⁶ and X⁷ are both —H or both methyl; and
X⁸ is fluoro, chloro, bromo or iodo;
or
(2) X⁴ is azepan-1-yl, morpholin-4-yl, pyrrolidin-1-yl, —N(CH₃)(CH(CH₃)₂) or 7-azabicyclo[2.2.1]heptan-7-yl; and R⁰ is 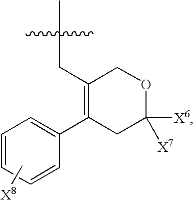
where X⁶, X⁷ and X⁸ are as above; or
(3) X⁴ is morpholin-4-yl or —N(CH₃)₂; and R⁰ is 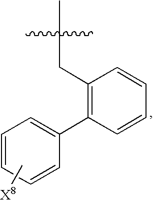
where X⁸ is as above;
or a pharmaceutically acceptable salt, prodrug, salt of a prodrug or metabolite thereof, in particulate form having a D₉₀ particle size not greater than about 3 μm; and pharmaceutically acceptable excipients including (a) at least one surfactant and at least one basifying agent and (b) at least one dispersant or bulking agent; said composition being dispersible in an aqueous medium to provide a suspension wherein the surfactant and basifying agent are in amounts that are effective together to inhibit particle size increase.

17. A process for preparing a pharmaceutical composition, comprising wet-milling an active pharmaceutical ingredient (API) in presence of at least one pharmaceutically acceptable basifying agent to a D₉₀ particle size not greater than about 3 μm to provide a milled drug substance; and suspending the milled drug substance in an aqueous medium with the aid of at least one pharmaceutically acceptable surfactant; wherein the at least one basifying agent and the at least one surfactant are present in the resulting suspension in amounts that are effective together to inhibit particle size increase; and wherein the API comprises a compound of Formula I 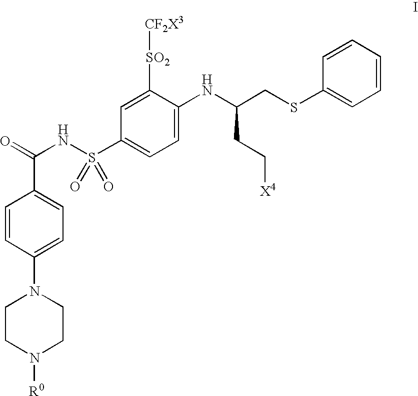
where
X³ is chloro or fluoro; and
(1) X⁴ is azepan-1-yl, morpholin-4-yl, 1,4-oxazepan-4-yl, pyrrolidin-1-yl, —N(CH₃)₂, —N(CH₃)(CH(CH₃)₂), 7-azabicyclo[2.2.1]heptan-7-yl or 2-oxa-5-azabicyclo[2.2.1]hept-5-yl; and R⁰ is 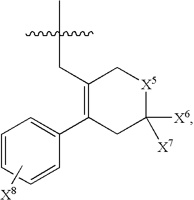
where
X⁵ is —CH₂—, —C(CH₃)₂— or —CH₂CH₂—;
X⁶ and X⁷ are both —H or both methyl; and
X⁸ is fluoro, chloro, bromo or iodo;
or
(2) X⁴ is azepan-1-yl, morpholin-4-yl, pyrrolidin-1-yl, —N(CH₃)(CH(CH₃)₂) or 7-azabicyclo[2.2.1]heptan-7-yl; and R⁰ is 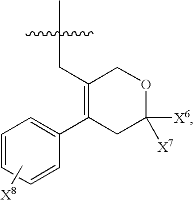
where X⁶, X⁷ and X⁸ are as above; or
(3) X⁴ is morpholin-4-yl or —N(CH₃)₂; and R⁰ is 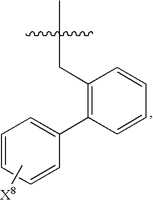
where X⁸ is as above;
or a pharmaceutically acceptable salt, prodrug, salt of a prodrug or metabolite thereof.

18. The process of claim 17, wherein the API comprises ABT-263 bis-HCl.

19. The process of claim 17, wherein the API is milled to a D₉₀ particle size not greater than about 800 nm and/or a D₅₀ particle size not greater than about 350 nm.

20. The process of claim 17, wherein the wet-milling comprises high-pressure homogenization.

21. The process of claim 17, wherein the at least one surfactant is added to the API and the at least one basifying agent before wet-milling.

22. The process of claim 17, wherein the at least one basifying agent comprises sodium bicarbonate.

23. The process of claim 17, further comprising adding a dispersant or bulking agent to the suspension and drying the suspension to provide a reconstitutable powder.

24. A method for treating a disease characterized by apoptotic dysfunction and/or overexpression of an anti-apoptotic Bc1-2 family protein, comprising administering to a subject having the disease a therapeutically effective amount of the composition of claim 1.

25. The method of claim 24, wherein the composition is administered parenterally or orally.

26. The method of claim 24, wherein the disease is a neoplastic disease.

27. The method of claim 26, wherein the neoplastic disease is selected from the group consisting of cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma and combinations thereof.

28. The method of claim 26, wherein the neoplastic disease is a lymphoid malignancy.

29. The method of claim 28, wherein the lymphoid malignancy is non-Hodgkin's lymphoma.

30. The method of claim 26, wherein the neoplastic disease is chronic lymphocytic leukemia or acute lymphocytic leukemia.

31. The method of claim 24, wherein the composition administered comprises ABT-263 free base, ABT-263 bis-HCl or a combination thereof.

32. The method of claim 31, wherein the composition is orally administered in a dose of about 50 to about 500 mg ABT-263 free base equivalent per day at an average treatment interval of about 3 hours to about 7 days.

33. The method of claim 31, wherein the composition is administered once daily in a dose of about 200 to about 400 mg ABT-263 free base equivalent per day.

34. A method for maintaining in bloodstream of a human cancer patient a therapeutically effective plasma concentration of ABT-263 and/or one or more metabolites thereof, comprising administering to the patient the composition of claim 8 in a dosage amount of about 50 to about 500 mg ABT-263 free base equivalent per day, at an average dosage interval of about 3 hours to about 7 days.
